(19) United States
(12) Patent Application Publication
(10) Pub. No.: US 2007/0184522 A1
(43) Pub. Date: Aug. 9, 2007

(54) Cell death modulation via antagonists of Fasl and Fas activation

(75) Inventors: Abdolreza Zarnegar, Gibsonia, PA (US); Marie C. DeFrances, Gibsonia, PA (US); Chun-Bin Zou, Pittsburgh, PA (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: University of Pittsburgh of the Commonwealth System of Higher Education, Pittsburgh, PA (US)

(21) Appl. No.: 11/113,951

(22) Filed: Apr. 25, 2005

Related U.S. Application Data
(60) Provisional application No. 60/565,283, filed on Apr. 23, 2004.

(51) Int. Cl. C12P 21/06; C07K 7/06; C07K 7/08; C07K 14/715

(52) U.S. Cl. 435/69.1; 530/350; 435/320.1; 435/325; 530/329; 530/330

(57) ABSTRACT

The invention provides a polypeptide that attenuates the activation of Fas, TNFR1, or both. The polypeptide can be used to treat conditions associated with dysregulation of the cell death or inflammatory pathway and can be formulated into pharmaceutical compositions for medical or veterinary use.

### CROSS-REFERENCE TO RELATED APPLICATIONS

This patent application claims the benefit of U.S. Provisional Patent Application No. 60/565,283 filed Apr. 23, 2004, the entirety of which is incorporated herein by reference thereto.

### STATEMENT REGARDING FEDERALLY SPONSORED RESEARCH AND DEVELOPMENT

This invention was made in part with Government support under Grant Number 1R01CA95782-03 awarded by NIH. The Government may have certain rights in this invention.

### BACKGROUND OF THE INVENTION

Programmed cell death, often referred to as apoptosis, is a cell suicide process that occurs in animal cells. Normally, cell death takes place during the growth and development of an organism, and it is used to eliminate excess cells. Cell death also is triggered when cells become damaged beyond normal repair mechanisms, or dangerous to the organism, such as autoimmune cells. Suppression, overexpression, or mutation of the genes that control the process, however, can lead to disease. More specifically, excessive cell death activity has been linked to liver disease, kidney disease, disorders of the pancreas, autoimmune diseases such as AIDS and rheumatoid arthritis, and neurodegenerative disorders such as Alzheimer's or Parkinson's. The ability to control and suppress overactive cell death would be a useful tool in the treatment of many serious diseases. (Thatte et al., 1997; Connolly et al., 2001).

Fas is a transmembrane cell surface receptor expressed in a variety of tissues and cell types and is activated by the binding of its cognate ligand known as Fas-Ligand (FasL). Fas is a member of the Tumor Necrosis Factor Receptor (TNFR) superfamily of apoptosis promoting cell surface transmembrane receptors. The binding of FasL to Fas results in receptor self-trimerization and clustering, which subsequently engages a cascade of biochemical events that culminates in apoptosis. The Fas/FasL system has been linked to human diseases, including liver and autoimmune diseases.

Met, a transmembrane cell surface receptor for hepatocyte growth factor, has the ability to bind the Fas receptor and prevent its activation. (Wang et al., 2002). Met is a heterodimer consisting of an alpha and a beta subunit linked together by a disulfide bond. The alpha chain is approximately 282 amino acids in length (amino acid residues 25-307) and remains entirely extracellular, while the beta chain is a single-pass transmembrane protein measuring approximately 1080 amino acid residues long (of which amino acids 308 to 932 remain extracellular) and harbors the tyrosine kinase and the associated signaling and regulatory domains within its intracellular cytoplasmic region. Relatively little or no information exists on the structure-function of the extracellular domain (the ectodomain) of Met besides its role in HGF binding. The mechanism by which Met binds Fas and inhibits activation of apoptosis is also unknown.

The ability to regulate members of the TNFR superfamily would be useful in establishing new methods of treating the variety of diseases associated with dysregulation of the cell death pathway.

### BRIEF SUMMARY OF THE INVENTION

The invention provides a polypeptide that attenuates the activation of one or more members of the TNFR superfamily particularly, Fas, TNFR1, or both. The inventive polypeptide can be used for preventing activation of the cell death pathway and can be used therapeutically in treating conditions, such as those described above, associated with aberrant activation of the cell death pathway by Fas or TNFR1. The invention also provides a pharmaceutical composition comprising the inventive polypeptide and a pharmaceutically acceptable carrier, which can be administered to facilitate treatment of such conditions. These and other advantages of the invention, as well as additional inventive features, will be apparent from the description of the invention provided herein.

### BRIEF DESCRIPTION OF THE DRAWINGS

FIGS. 1a-1g present data demonstrating that the alpha subunit of Met (AlphaMet) interacting with the extracellular part of Fas via novel motif YLGA (SEQ ID NO:1). The Met alpha but not beta chain binds to Fas (FIG. 1a). Various regions of the mouse Met cDNA corresponding to the alpha chain or the extra-cellular portion of the Met beta chain were cloned into expression vectors (pCRT7 TOPO/AlphaMet or pCRT7 TOPO/beta chain) and subjected to in vitro transcription/translation by a rabbit reticolucyte lysate translation system. Empty vector was included in each experiment as a control. The expressed proteins were labeled with ³⁵S-Methionine. Pull down assays were performed using Fas-Fc, a chimeric molecule which lacks the interacellular cytoplasmic domain and protein-A agarose as described in Wang et al., 2002. FIG. 1b is a schematic presentation of the Met alpha chain and its mutant version used to map the region interacting with Fas. The C-terminus of each construct contains a hexa-Histidine tag for ease of detection. Start cordon ATG was added to the N-terminally truncated constructs. The YLGA (SEQ ID NO:1) motif mutation was prepared by PCR, in which YLGA (SEQ ID NO:1) was replaced by DHER (SEQ ID NO:24). FIG. 1c presents data demonstrating that the N-terminal 100 amino acids of the Met alpha subunit are sufficient to bind Fas. In vitro translated ³⁵S-methionine labeled truncated/mutated AlphaMet proteins were pulled down with 2 μg of Fas-Fc or the same amount of IgG. Lane 1: 10% of AlphaMet input; Lane 2: AlphaMet with IgG control; Lanes 3 to 8: pull down of various truncated AlphaMet proteins; N-terminal 1-106 residues; C-terminal truncated, residues 1-210; alpha chain, residues 1-306; alpha chain with transmembrane domain, 1-338; N-terminally truncated alpha chain, residues 100-306; and N-terminally truncated with addition of the transmembrane domain 100-338, respectively. FIG. 1d depicts the amino acid sequence alignment of alpha chain with FasL, determined using the CLUSTALW, SIM+LALNVIEW, and ALIGN at ExPASy Proteomic Tools (available on the internet at us.expresly.org/tools). The numbers indicate the amino acid residues of the indicated protein. FIG. 1e presents data demonstrating that wild type AlphaMet but not the YLGA mutant binds to Fas. Recombinant His-tagged AlphaMet or its mutant version were expressed in E. coli, purified with chromatography, and subjected to Fas-Fc (lanes labeled Fas) or control IgG pull down experiments, and detected by Western blot using anti-His antibody that recognizes C-terminal His. This antibody also recognizes the Fas-Fc which is also tagged at the C-terminus by His. FIG. 1f presents data demonstrating that AlphaMet binds to the endogenous Fas from Jurkat and Hepa 1-6 cells. Purified recombinant AlphaMet protein (approximately 10 μg) was mixed with Hepa 1-6 cells or Jurkat cell lysates and subjected to immunoprecicitation using anti-Fas antibody or control IgG followed by Western blot using anti His-tag antibody as a probe. FIG. 1g presents data demonstrating that synthetic peptides derived from mouse or human AlphaMet bind to Fas with high affinity as determined, by ELISA. Peptides (1 μg/ml) were coated into the 96-well microtiter plates in triplicate and after blocking with 1% BSA, increasing amounts of Fas-Fc were added as indicated. The control peptide was a scrambled 13 mer. After washing the wells, anti-human IgM-HRP conjugates (1:20,000) were added to detect the bound Fas-Fc using TMD dye and the OD value was determined at a wavelength of 450 nm. One-way ANOVA was carried out for determination of statistical significance.

FIGS. 2a-2e present data demonstrating that AlphaMet competes with Fas-ligand for Fas binding. FIG. 2a presents data demonstrating that recombinant purified His-tagged AlphaMet (approximately 5 μg) incubated with Fas-Fc in the presence or absence of FasL or unrelated protein (albumin, FIG. 2c), subjected to pull down experiments followed by Western blot using anti-His antibody that recognizes C-terminal His. This antibody also recognizes Fas-Fc which is also tagged at the C-terminus by His. In FIG. 2b, a fixed amount of purified recombinant Fas-ligand was added to Fas-Fc in the absence or presence of increasing amounts of AlphaMet as indicated. Samples were subjected to pull down using proteinA agarose followed by Western blot using a polyclonal rabbit anti-FasL antibody. FIG. 2d shows the results of ELISA, carried out as described above. Fas ligand (1 μg/ml) was coated in the wells of a microtiter plate (triplicate wells) then 1 μg/ml of Fas-Fc was added in the absence or presence of an increasing amount of the indicated polypeptide. A 13 mer YLGA polypeptide derived from FasL also was synthesized and used. The control peptide was a scrambled 13 mer. Anti human IgM-HRP conjugates were used to detect the bound Fas-Fc. Since human AlphaMet sequence in the YLGA (SEQ ID NO:1) motif contains the amino acid phenylalanine (F) instead of tyrosine (Y) (FLGA (SEQ ID NO:32) instead of YLGA (SEQ ID NO:1)) and that these two amino acids are identical in structure to each other, a FLGA (SEQ ID NO:32) tetramer synthetic peptide also was tested in the ELISA assays and found that FLGA (SEQ ID NO:32) binds to Fas with similar affinity as that of YLGA (SEQ ID NO:1) tetramer. One-way ANOVA was carried out for determination of statistical significance. FIG. 2e presents data demonstrating that AlphaMet and YLGA polypeptides inhibit FasL binding to Fas in Jurkat cells. Cells (3×10⁵) were plated in 24-well plates having Poly-L-Lysine pre-treated cover glass. 2 μg/ml of Fas ligand was added and the cells were incubated for 30 min on ice. Cells were then washed by PBS twice, and were subjected to immunostaining using anti-Fas ligand antibody and fluorescent microscopy. In Frame A, Fas ligand was not added, in B, only Fas ligand was added, in C-F, Fas ligand was added with different polypeptides as follows: AlphaMet, 12 mer YLGA, control peptide, and YLGA mutated AlphaMet, respectively.

FIGS. 3a-3f present data demonstrating that AlphaMet prohibits Fas homo-trimerization and Fas microaggregation. FIG. 3a presents data collected from Jurkat cells (1×10⁶) treated with 2 μg/ml of Fas Ligand with or without AlphaMet for 30 minutes on ice. Cells were washed with PBS twice and then incubated with 2 mM of DTSSP in PBS for 30 minutes. The crosslinking process was stopped by 20 mM of Tris (pH 7.5) for another 30 minutes. Cells were spun down and washed with PBS twice and lyzed in RIPA buffer. Cell lysates were subjected to SDS-PAGE under non-reducing or reducing conditions (FIG. 3b) and Western blot analysis using the mouse monoclonal antibody against human Fas. Fas monomer migrating as a doublet as well as trimerized Fas are indicated in the figure. FIG. 3c depicts the quantification of the data in FIG. 3a by densitometry. The data are the mean and SE from three different independent crosslinking and Western blot experiments. FIG. 3d shows a FRET analyses of Hepa 1-6 transfected with Fas-fluorescent protein chimeric proteins (CFP-Fas and YFP-Fas). Frame A: Acceptor CFP-Fas image; Frame B: Acceptor pre-bleach CFP-Fas image; Frame C: Acceptor post-bleach image. Frame D: Donor YFP-Fas image; Frame E: Donor pre-bleach image; Frame F: Donor pre-bleach image. The CFP-Fas strength in the selected region obtained a gain of 19.3% efficiency in the presence of FasL for 15 minutes. No FRET was observed without FasL. In the presence of FasL and AlphaMet, no significant FRET could be detected. FIG. 3e depicts the results of experiments in which CFP-tagged Fas or CFP control vector were transiently expressed in Hepa 1-6 Cells (3×10⁵). Cells were treated with 1 μg/ml of Fas ligand or 100 ng/ml of Jo2 with or without 5 μg/ml of AlphaMet or 1 μg/ml of YLGA 12 mer for 30 minutes on ice. Cells were fixed and analyzed by fluorescence microscopy. Frame A: AlphaMet treatment only, no FasL; Frame B: Fas ligand treatment only; Frame C: Fas ligand and AlphaMet treatment; Frame D: Fas ligand and 12 mer YLGA; Frame E: Jo2 treatment only and Frame F: Jo2 and AlphaMet treatment. The percentage cells demonstrating capping of Fas was calculated by enumerating at least 300 cells from random fields and the data were quantified and depicted graphically in FIG. 3f.

FIGS. 4a-4d present data demonstrating that AlphaMet and YLGA polypeptide attenuate apoptosis in Jurkat and Hepa 1-6 cell lines. For FIG. 4a, Jurkat cells (1.5×10⁵) were overnight treated in triplicate with 5 ng/ml of crosslinked human Fas ligand with or without 5 μg/ml of recombinant AlphaMet or 1 μg/ml of the indicated polypeptides. Percent apoptosis was determined by Trypan Blue staining using a hemocytometer. The data are representative and were repeated at least four times. FIG. 4b presents data demonstrating that cleaved Caspase 3 fragment was detected in apoptotic Jurkat cells by specific monoclonal antibody. The 17 kDa fragment of cleaved Caspase 3 is visualized by this antibody. The blot was reprobed for β-actin as a loading control. For FIG. 4c, serum deprived Hepa 1-6 cells (3×10⁵) were overnight treated with 1 μg/ml of crosslinked mouse Fas ligand with or without AlphaMet or the indicated polypeptide. Media were collected and centrifuged to pellet the dead cells. The media (50 μl) were subjected to LDH activity assay as suggested by the manufacturer. The data in FIG. 4c represents three independent experiments. FIG. 4d presents the results of experiments demonstrating that cleaved Caspase 3 fragment was detected in apoptotic Hepa 1-6 cell lysates.

FIGS. 5a-5c presents the results of an experiment demonstrating protection of the liver by AlphaMet and the corresponding polypeptides from Fas-induced apoptosis in Balb/c Mice. For FIG. 5a, Balb/c mice (10 per group) were injected with Jo2 (2.5 μg/g of mouse body weight) in the presence of AlphaMet or polypeptides as indicated, and analyzed for the extent of hepatocyte apoptosis and liver damage by immunohisological and biochemical techniques. The percentage of TUNEL positive was determined in multiple liver lobes of each liver in random fields counting at least 500 hepatocytes in a double-blind manner. Frame A: Normal mouse liver; Frame B: Jo2 treated mouse liver; Frame C: Jo2 plus AlphaMet treated mouse; Frame D: Jo2 plus YLGA 12 mer; Frame E: Jo2 and YLGA tetramer treatment; Frame F: Jo2 and YLGA mutated AlphaMet, respectively. These studies were repeated three times with identical outcome. FIG. 5b graphically presents the quantification of hepatocyte apoptosis TUNEL positive nuclei. The data were analyzed by One-way ANOVA and P<0.05 was considered significant. For FIG. 5c, an equal amount of livers were subjected to Caspase 3 activity assay using DEVD-AFC as a substrate and DEVD-CHO as an inhibitor, as recommended by the manufacturer. The data are the mean and SE and were repeated in three independent experiments. The data were analyzed by One-way ANOVA and P<0.05 was considered significant.

### DETAILED DESCRIPTION OF THE INVENTION

The invention provides a synthetic polypeptide that attenuates the activation of a member of the TNFR superfamily, which includes Fas, TNFR1, and other proteins. More preferably, the polypeptide attenuates the activation of both Fas and TNFR1. The inventive polypeptide attenuates the activation of Fas, TNFR1, or other members of the TNFR superfamily in that it reduces the activation of such protein to a measurable extent. Attenuation can be assessed using in vitro assays (e.g., pull down experiments as described below, immunohistological techniques such as ELISA, Western blotting, immunoprecipitation, etc.). Activation of Fas, TNFR1, or both (or other members of the TNFR superfamily) can also be attenuated in vivo. For example, a cell line can be transfected with a plasmid containing the polypeptide. Apoptosis can then be induced by the addition of FasL, followed by a flow cytometry analysis of the cells.

The inventive polypeptide can attenuate the activation of the members of the TNFR superfamily (e.g., Fas, TNFR1, or both) to a varying degree, depending on which member of the TNFR superfamily is to be affected and also on the exact sequence of the polypeptide. Desirably, however, the inventive polypeptide attenuates activation of the members of the TNFR superfamily to a degree sufficient to modulate apoptosis. In preferred embodiments, the inventive polypeptide blocks or substantially blocks activation of, for example, Fas, TNFR1, and preferably both. Where the degree of attenuation of the activity of such proteins can be quantified, desirably the inventive polypeptide attenuates the activity of members of the TNFR superfamily (especially Fas, TNFR1, or both) by at least about 70%, such as at least about 80% or even at least about 90%). Most desirably, the inventive polypeptide blocks or substantially blocks the activation of the member of the TNFR superfamily, such as, for example, attenuating the activation of Fas, TNFR1, or both (or other member of the TNFR superfamily) by at least about 90% or even above about 98% or 99%.

The inventive polypeptide comprises at least four or at least five or at least six, such as at least twelve or at least about 36 amino acids. The inventive polypeptide can also contain up to several hundred amino acids, such as having up to about 100 or up to about 200 or up to about 300 amino acids. The inventive polypeptide can include longer amino acid sequences than these, but such is not typical. In one embodiment inventive polypeptide can comprise, consist of, or consist essentially of the alpha domain of Met, or C-terminal truncations of AlphaMet, the sequence of which is known (Stamos et al., 2004). Three examples of such truncations comprise amino acids 1-106, amino acids 1-210, or amino acids 1-306. The inventive polypeptide is, however, a synthetic sequence in that it does not include the entire sequence of AlphaMet amino acids (indeed, the three examples just listed are C-terminal deletions). The inventive polypeptide also can comprise a truncation of a Semaphorin and Plexin. Indeed, the YLGAV (SEQ ID NO:5) corresponding to PlexinA4 is identical to the YLGAV (SEQ ID NO:5) present in the Fas binding domain of FasL. FLGAV (SEQ ID NO:37) the human counterpart of YLGAV (SEQ ID NO:5) also is very active in binding to Fas and inhibiting FasL binding to Fas and hence inhibiting Fas activation. In other embodiments, the inventive polypeptide is a shorter polypeptide, such as consisting of from about 5 to about 50 amino acids (e.g., less than about 15, and preferably less than about 12 or less than about 10 amino acids; or between about 4-6 amino acids, about 10-15 amino acids, about 15-20 amino acids, about 20-25 amino acids, about 25-30 amino acids, about 30-35 amino acids, or about 30-40 amino acids). In the experimental examples set forth herein, a 4-mer (YLGA SEQ ID NO:1 or its human counterpart FLGA (SEQ ID NO: 32), 12-mers (HHIYLGATNYIY, SEQ ID NO:26) and (HHIFLGATNYIY, SEQ ID NO:33), a 13-mer (AHSSYLGAVFNLT SEQ ID NO:30) and a 36-mer (FTAETPIQNVVLHGHHIYLGATNYIYVLNDKDLQKV, SEQ ID NO:25) were employed. Another polypeptide (TGHIYLGAVNRIY SEQ ID NO:31) is another specific example of the inventive polypeptide.

Preferred examples of the inventive polypeptide comprise, consist of, or consist essentially of the 4 contiguous amino acid sequences YLGA (SEQ ID NO:1), FLGA (SEQ ID NO: 32), YLGG (SEQ ID NO:2), or FLGG (SEQ ID NO:34). In other examples, the inventive polypeptide comprise, consist of, or consist essentially of the following 5 contiguous amino acid residues having the sequence IYLGA (SEQ ID NO:3), IYLGG (SEQ ID NO:4), YLGAV (SEQ ID NO:5), YLGGV (SEQ ID NO:6), IFLGA (SEQ ID NO:35), IFLGG (SEQ ID NO:36), FLGAV (SEQ ID NO:37), or FLGGV (SEQ ID NO:38), or the following 6 contiguous amino acid residues having the sequence IYLGAV (SEQ ID NO:7), IYLGGV (SEQ ID NO:8), IFLGAV (SEQ ID NO:39), or IFLGGV (SEQ ID NO:40). The inventive polypeptide is not limited to the amino acid sequences described above and can include conservative substitutions or other changes that retain the ability of the inventive polypeptide to attenuate the activation of the member of the TNFR superfamily (e.g., Fas or TNFR1, or both).

The inventive polypeptide can be prepared by methods known to those of ordinary skill in the art. For example, the inventive polypeptide can be synthesized using solid phase polypeptide synthesis techniques (e.g. Fmoc). Alternatively, the polypeptide can be synthesized using recombinant DNA technology (e.g., using bacterial or eukaryotic expression systems). Accordingly, to facilitate such methods, the invention provides genetic vectors (e.g., plasmids) comprising a sequence encoding the inventive polypeptide, as well as host cells comprising such vectors. Furthermore, the invention provides the inventive polypeptide in recombinant form.

However it is made, the inventive polypeptide can be isolated and/or purified (or substantially isolated and/or substantially purified). Accordingly, the invention provides the inventive polypeptide in substantially isolated form. The polypeptide can be isolated from other polypeptides as a result of solid phase protein synthesis, for example. Alternatively, the polypeptide can be substantially isolated from other proteins after cell lysis from recombinant production. Standard methods of protein purification (e.g., HPLC) can be employed to substantially purify the inventive polypeptide.

The invention provides a preparation of the inventive polypeptide in a number of formulations, depending on the desired use. For example, where the polypeptide is substantially isolated (or even nearly completely isolated from other proteins), it can be formulated in a suitable medium solution for storage (e.g., under refrigerated conditions or under frozen conditions). Such preparations can contain protective agents, such as buffers, preservatives, cryprotectants (e.g., sugars such as trehalose), etc. The form of such preparations can be solutions, gels, etc., and the inventive polypeptide can, in some embodiments, be prepared in lyophilized form. Moreover, such preparations can include other desired agents, such as small molecules or even other polypeptides and proteins, if desired. Indeed, the invention provides such a preparation comprising a mixture of different embodiments of the inventive polypeptide (e.g., a plurality of polypeptide species as described herein).

The invention also provides a pharmaceutical composition comprising of one or more of the inventive polypeptides (including mixtures thereof) and a pharmaceutically acceptable carrier. Any carrier which can supply the polypeptide without destroying the vector within the carrier is a suitable carrier, and such carriers are well known in the art. The composition can be formulated for parenteral, oral, or topical administration. For example, a parenteral formulation could consist of a prompt or sustained release liquid preparation, dry powder, emulsion, suspension, or any other standard formulation. An oral formulation of the pharmaceutical composition could be, for example, a liquid solution, such as an effective amount of the composition dissolved in diluents (e.g., water, saline, juice, etc.), suspensions in an appropriate liquid, or suitable emulsions. An oral formulation could also be delivered in tablet form, and could include excipients, colorants, diluents, buffering agents, moistening agents, preservatives, flavoring agents, and pharmacologically compatible excipients. A topical formulation could include compounds to enhance absorption or penetration of the active ingredient through the skin or other affected areas, such as dimethylsulfoxide and related analogs. The pharmaceutical composition could also be delivered topically using a transdermal device, such as a patch, which could include the composition in a suitable solvent system with an adhesive system, such as an acrylic emulsion, and a polyester patch.

The invention also provides a method of employing the inventive polypeptide to attenuate the activation of one or more members of the TNFR superfamily, desirably Fas and/or TNFR1. Such method can be employed, for example, to inhibit cell death (e.g., apoptosis) or an inflammatory response in cells and tissues, and it can be employed in vivo, ex vivo or in vitro. Thus, the invention provides for the use of the inventive polypeptide for attenuating cell death in accordance with such methods. For in vitro application, the inventive polypeptide is provided to cells, typically a population of cells (e.g., within a suitable preparation, such as a buffered solution) in an amount and over a time course sufficient to inhibit apoptosis within the cells or to inhibit inflammation. If desired, a controlled population untreated with the inventive polypeptide can be observed to confirm the effect of the inventive polypeptide in reducing the inhibition of cell death or inflammation within a like population of cells.

For in vivo use, the inventive polypeptide can be delivered to a human or animal subject in an amount and at a location sufficient to inhibit or attenuate apoptosis or inflammation within the patient (e.g., within desired tissue). The inventive polypeptide can be formulated into a suitable pharmaceutical composition (e.g., as described above or as otherwise known to those of ordinary skill in the art) for delivery into the subject. The delivery can be local (e.g., by injection or implantation within the desired tissue to be treated) or systemic (e.g., by intravenous or parenteral injection). The example set forth below demonstrates that systemic delivery of the inventive polypeptide can be employed to attenuate apoptosis in liver tissue. Thus, the inventive polypeptide can be employed (alone or adjunctively with other treatments) to treat diseases or disorders of the liver, such as liver failure or hepatitis, in patients in need of such treatment. However, the inventive polypeptide also can be used (alone or in conjunction with other modes of treatment) to treat patients suffering from other diseases or disorders mediated by cell death or apoptosis and tissue inflammation, such as liver disease, kidney and lung diseases, disorders of the pancreas, autoimmune diseases such as AIDS and rheumatoid arthritis, and neurodegenerative disorders such as Alzheimer's, Parkinson's or spinal cord injury.

Accordingly, the invention provides a method for treating patients suffering from such diseases or disorders and in need of treatment. In accordance with the inventive method, a pharmaceutical composition comprising at least one species of inventive polypeptide is delivered to such a patient in an amount and at a location sufficient to treat the disorder or disease. The invention also provides for the use of the inventive polypeptide for preparing a medicament to treat such disorders. As noted, the inventive polypeptide (or pharmaceutical composition comprising such) can be delivered to the patient systemically or locally, and it will be within the ordinary skill of the medical professional treating such patient to ascertain the most appropriate delivery route, time course, and dosage for treatment. It will be appreciated that application of the inventive method of treating a patient most preferably substantially alleviates or even eliminates such symptoms; however, as with many medical treatments, application of the inventive method is deemed successful if, during, following, or otherwise as a result of the inventive method, the symptoms of the disease or disorder in the patient subside to a degree ascertainable.

### EXAMPLES

The following examples further illustrate the invention but, of course, should not be construed as in any way limiting its scope. These examples demonstrate that the alpha subunit (amino acids 1-306) of Met specifically binds to Fas and that this binding is facilitated by a YLGA (SEQ ID NO:1) or a FLGA (SEQ ID NO:32) amino acid motif located in the N-terminal region of the Met alpha chain. These examples also demonstrate that the binding of the alpha subunit of Met to Fas is abrogated by the addition of increasing amounts of FasL. The experimental data reveal that polypeptides having a sequence similar to that of the conserved region of AlphaMet, FasL, Met related family members such as Plexin 4A, or the TNF family members are able to block the activation of Fas or TNFR1 by preventing the receptors from self-trimerization, clustering, and capping. In addition, these examples demonstrate that the binding of Fas to FasL is abrogated by increasing amounts of polypeptide corresponding to the YLGA (SEQ ID NO:1) or FLGA (SEQ ID NO:32) motif of AlphaMet, or the YLGA (SEQ ID NO:1) motif of FasL. These examples also demonstrate that polypeptides of the invention suppress the ability of Fas to self-trimerize and cluster. These examples additionally demonstrate the ability of the inventive polypeptides to block Fas ligand-induced apoptosis in Jurkat cells (expressing low levels of Met) and Hepa 1-6 cells (expressing high levels of Met). These examples also demonstrate that the activation of Caspase 3 (a downstream executioner in Fas mediated apoptosis) was reduced by the inventive polypeptide. Also, this example demonstrates that Fas-induced apoptosis of the liver in mice is greatly reduced by treatment with the inventive polypeptide or AlphaMet.

Many procedures, such as Western blots, PCR, vector construction, including direct cloning techniques (including DNA extraction, isolation, restriction digestion, ligation, etc.), cell culture, transfection of cells, protein expression and purification, and ELISA and immunological assays are techniques routinely performed by one of ordinary skill in the art (see generally Sambrook et al., Molecular Cloning, A Laboratory Manual, Cold Spring Harbor Laboratory, Cold Spring Harbor, N.Y. 1989). The following sections describe particular Materials and Methods used in this example.

Cell lines, chemicals and antibodies. Jurkat cells were cultured in RPMI 1640 medium complemented with 10% of FBS. Hepa 1-6 and HepG2 cell lines were cultured in DMEM complemented with 10% FBS. Anti-Fas antibodies, anti-Fas ligand antibody, anti-Met antibodies, and Protein A Agarose beads were from Santa Cruz and UpState Biotechnology. Anti-C-terminal His-tag antibody was from Invitrogen. Fas-Fc chimera protein, soluble Fas Ligand, and its crosslinking antibody were purchased from R&D. Anti-human IgM-HRP conjugates were from Chemicon. Chemical crosslinker 3,3′dithiobis [sulfosuccinimidylpropionate] (DTSSP), ELISA color reagent 3,3′,5,5′ Tetramethylbenzidine (TMB), Lyzozyme, protease inhibitor cocktail, and DEAE column were from Sigma. Nickel-beads for protein purification were from Invitrogen. Isotopes ³⁵S labeled methionine and Cystine were from Amersham Biosciences. Synthetic polypeptides were made by Genemed Synthesis, Inc., CA. All other chemicals used in the experiments were commercially available with the highest quality.

Plasmid construction. Mouse origin MET cDNA was used as a PCR template, and a variety of forward (included ATG start codon) and reverse primers were designed based on the mouse C-MET gene sequence: Forward1, 5′-ATGAAGGCTCCCACCGTG (SEQ ID NO:9); Forward100, 5′-ATGGGGACTGCAGCAGCAAAG (SEQ ID NO:10); Forward307, 5′-ATGCCACAAGGGAAGAAGTG (SEQ ID NO:11); Reverse106, 5′-CTTTGCTGCTGCAGTCCCG (SEQ ID NO:12); Reverse210, 5′-CAGTGAATAACCAGGAGG (SEQ ID NO:13); Reverse306, TCTCTTCCTT CTTTTTTCTGTCAG (SEQ ID NO:14); Reverse929, 5′-ATTCTGATCCGGTTGAACG (SEQ ID NO:15). YLGA was mutated by PCR, with the BamH I overhang site primers as follows: Forward1, 5′-ATGAAGGCTCCCACCGTG (SEQ ID NO:16); Reverse YLGA 5′-CGGGATCGATCACGAACGCACAAACTACATTTATG (SEQ ID NO:17); Forward YLGA, 5′-CGGGGATCCATGGCCGTGTAGGACGACATTCTGG (SEQ ID NO:18); Reverse106, 5′-CTTTGCTGCTGCAGTCCCG (SEQ ID NO:19). Two PCR fragments were digested with BamH I and ligated into one fragment. PCR products were inserted into the pCRT7 TOPO vector (Invitrogen), and the DNA inserts were confirmed by DNA sequencing. C-terminal tagged polyhistidine (6×His) DNA constructs were produced by PCR using pCRT7 TOPO inserts as templates. These DNA fragments were cloned into the PCR3.1 (Invitrogen) plasmid using the following primers: Forward1, 5′-ATGGAGGCTCCCACCGTG (SEQ ID NO:20); Forward106, 5′-ATGGGGACTGCAGCAGCAAAG (SEQ ID NO:21); Forward307, 5′-ATGGCCACAAGGGAAGAAGTG (SEQ ID NO:22); ReverseHis, 5′-TCAATGGTGATGGTGATGATGACCGG (SEQ ID NO:23).

In vitro translation and pull down experiments. The expression plasmid pCRT7 TOPO constructs were used as templates. A TNT coupled Reticulocyte lysate system (Promega) was used to translate a variety of α-Met truncates and the extracellular portion of the β-chain. ³⁵S Methionine and Cystine were used to label the translated protein. Synthesized protein was pre-cleared with Protein A-agarose. Fas-Fc was incubated with RIPA buffer diluted reticulocyte lysate at 4° C. for 2 hours. Protein-A agarose beads were then added into the mixture and rotated for another 2 hours. The mixture was spun down and washed with RIPA buffer 3 times. The beads with 30 μl of gel loading buffer were heated at 95° C. for 5 minutes and subjected to SDS-PAGE followed by autoradiography.

Protein expression and purification. Expression plasmid pCRT7 TOPO constructs were transformed into E. coli BL21 (DE3) competent cells (Invitrogen). E. coli were cultivated in 2% glucose LB at 37° C. and induced by IPTG at an OD value of 0.6. After being shaken at 25° C. for another 2 hours, the cells were harvested and lysed by Lysozyme, then repeatedly frozen and thawed in liquid nitrogen. The cell lysate was cleared by centrifugation and the supernatant was subjected to nickel-bead column purification. The elution was dialyzed and the protein was further purified by DEAE column. The purity of the purified recombinants was 90%.

Immunostaining. Jurkat cells were cultured overnight at a density of 10⁶ per ml in the 24-well plates that contained cover glass (pretreated with 0.1% of poly-L-Lysine solution). Cells were treated with Fas ligand with or without AlphaMet, or the synthesized polypeptides at 4° C. for 30 minutes. Cells were then washed with PBS pH 7.4 twice, fixed with cold methanol for 15 minutes, and blocked by incubating with blocking solution (5% donkey serum in PBS) for 1 hour. Anti-Fas ligand antibody (at a concentration of 1:1000) was diluted in blocking buffer, and incubated with cells for one additional hour. Cells were washed three times with PBS for 5 minutes each time followed by donkey anti-Rabbit Cyn3 conjugate (final concentration was 1 μg/ml) for 60 minutes. Cells were washed three times with PBS, for 10 minutes each and mounted.

Chemical crosslinking. Jurkat cells (1×10⁶) were treated with Fas Ligand with or without AlphaMet or a given polypeptide for 30 minutes on ice. Cells were washed with PBS twice and then incubated with 2 mM of DTSSP in PBS for 30 minutes. Crosslinking was stopped by 20 mM of Tris (pH 7.5) for another 30 minutes. Cells were spun down and washed with PBS twice and lyzed in RIPA buffer with or without DTT.

Apoptosis assay. Pre-washed Jurkat cells in RPMI 1640 medium containing 1% FBS were inoculated in 96-wells at a density of 10⁵ cells per well. Apoptosis was induced by cross-linked human Fas-ligand (R&D Systems) at a concentration of 0.5 ng/ml or Anti-Fas (human, activating) clone CH11 (Upstate biotechnology) at a concentration of 500 ng /ml. Hepa 1-6 cells were overnight starved before induction of apoptosis. The apoptosis in Hepa 1-6 was induced by anti-mouse Fas antibody Jo2 (200 ng/ml) or human recombinant Fas-ligand. The extent of cell viability and apoptosis was determined by several methods such as Trypan Blue stain, flow cytometry, LDH assay, and Hoechst staining.

Hepa 1-6 cell transfection. pCR3.1/inserts plasmids were transfected into Hepa 1-6 cells by the lipofectamine method (Invitrogen). Hepa 1-6 cells were plated into 6-well plates until the cells were 80% confluent, 2 μg of plasmid DNA was premixed with lipofectamine in a volume of 200 μl for 30 min, then the mixture was added into the pre-washed cells with 0.8 ml of serum-free medium. After the cells were cultivated at 5% CO₂, 37° C. for 5 hours, FBS was added in a final concentration of 10%. Transient transfected cells were lysed by RIPA buffer. The cell lysate were subjected to Western blotting, immunoprecipitation, and apoptosis.

Animal studies. Male Balb/c mice weighing 30 grams were purchased from Jackson Laboratory and used according to an IACUC approved protocol. Induction of liver apoptosis and determination of Caspase 3 activity and histological evaluation were performed as described previously (Wang et al., 2002). Animals (10 per group) were anesthetized by isoflurane and injected with Jo2 (Pharmingen 2.5 microgram/gram body weight) with or without AlphaMet (100 micrograms recombinant AlphaMet or 300 micrograms of synthetic YLGA polypeptides) in a total of 100 micro liters of saline per mouse. Control groups (N=10 per group) consisted of saline only, Jo2 only, and Jo2 plus mutated AlphaMet or scrambled polypeptide. During the experiment which lasted approximately 6 hours, animals were closely monitored every 10 to 15 minutes and if they appeared to be moribund were immediately sacrificed and liver tissue and was removed for biochemical and histological studies. To minimize animal suffering we did not do survival studies and death was never an end point in our study.

Statistical Analysis. Analysis of Variance (One-way ANOVA) and the Student t-test were utilized for determining the statistical significance of the data.

ELISA. Synthesized polypeptides (Genemed Synthesis, Inc. South San Francisco, Calif.) were dissolved in water and diluted in the coating buffer (0.05M of Sodium Carbonate, 0.02% of Sodium Azide, pH 9.6) to a final concentration of 1 ug/ml. One hundred microliter aliquots were added to coat each well of a 96-well microtitre plate. The coated plates were then washed with PBS-Tween (0.5%) three times. The plates were blotted and dried by tapping upside down on tissue paper. They were blocked using 200 μl of 1% of BSA in PBS solution followed by a 2 hour incubation. Plates were washed another three times, Fas-Fc was added, and the plates were incubated for another 2 hours. The plates were washed three times and anti-human IgM Fc HRP conjugates were included at a final concentration of 1:20,000, and incubated for 1 hour. Plates were washed three times again and ready-to-use TMD was added and incubated for 30 minutes. The color reaction was stopped by 2M of Sulfuric Acid. Plates were applied to color read at a wave length of 450 nm.

FRET assay. The CFP and YFP-tagged Fas mammalian expression vectors and their counterpart control vectors were a generous gift from Dr. Richard Siegel at NIH, Bethesda. These vectors have been previously used by Siegel et al., 2000, in FRET studies to demonstrate Fas self-assembly. Hepa 1-6 Cells were transiently transfected with one or two of these plasmids in combination or with control CYP/CFP vector lacking Fas molecule by the previously described method. After overnight culture, the transfected cells were treated with Fas ligand with or without AlphaMet. Cells were fixed by cold Methanol and applied to Fret analysis. Imaging was performed with Leica Confocal systems. FRET imaging acquisition and data analysis were performed with LCS Microlab software. Pre- and post-bleaching image sets of both donor (CFP) and acceptor (YFP) were acquired at the laser wave length of 473 nm or 517 nm correspondingly. CFP signals outlining the cell membrane were selected as the region of interest. Fret efficiency (E) was calculated as FRET_{eff}=(Dₚₒₛₜ−Dₚᵣₑ)/Dₚₒₛₜ (Dₚₒₛₜ and Dₚᵣₑ are the mean emission intensity prior to and following YFP bleaching).

### Example 1

This example demonstrates that the alpha subunit of Met (AlphaMet) specifically binds to Fas and a YLGA motif plays a pivotal role in Met and Fas interaction.

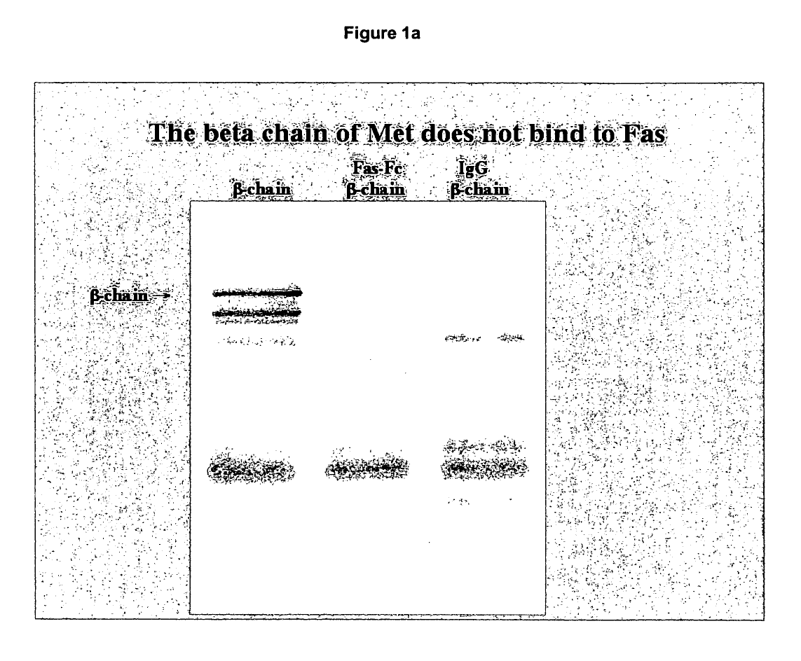

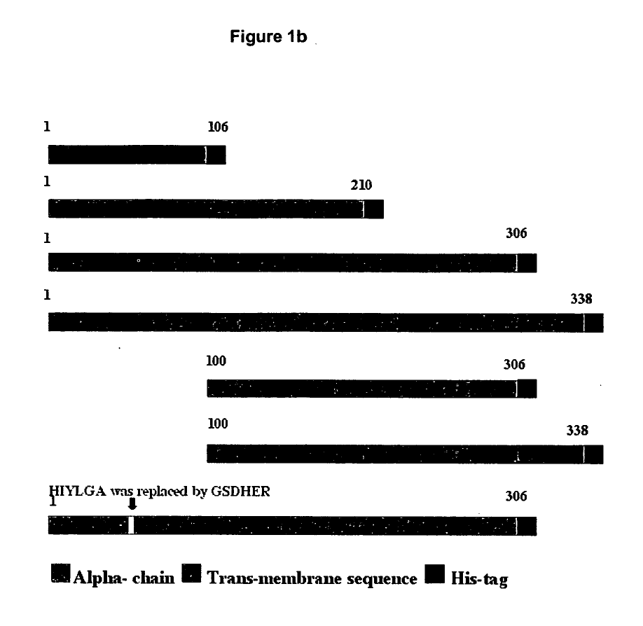

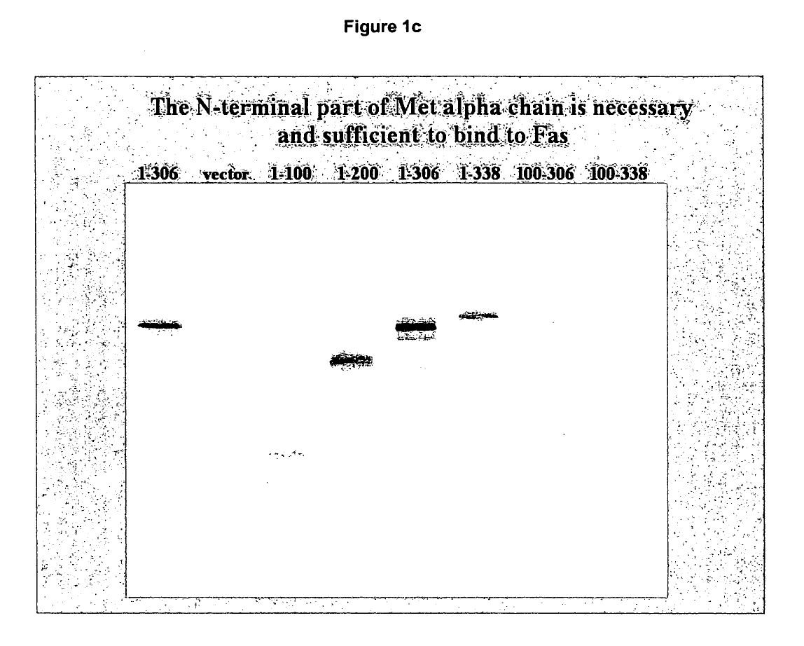

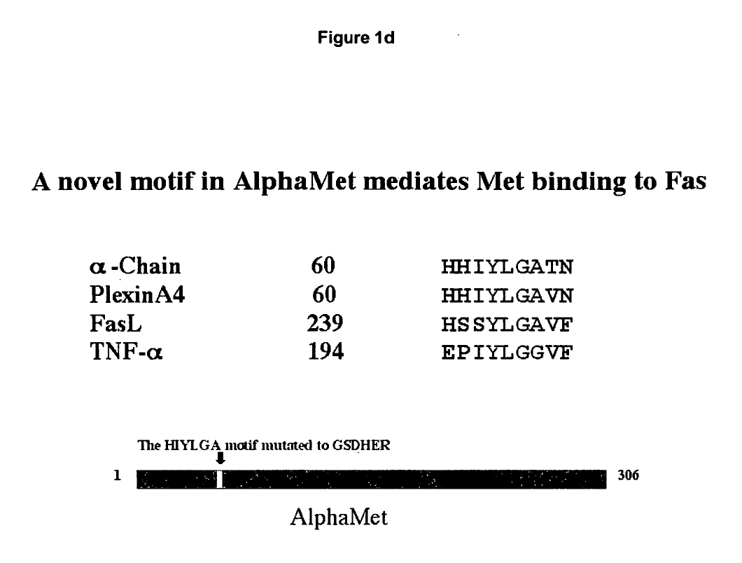

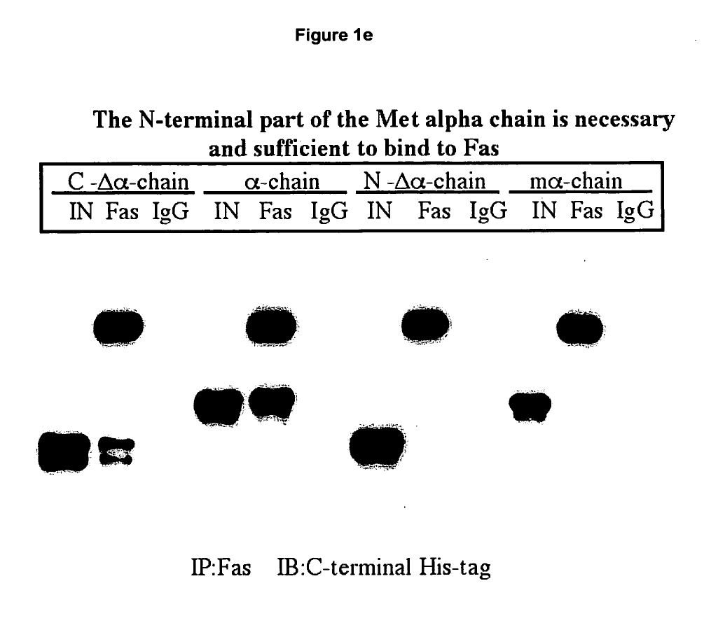

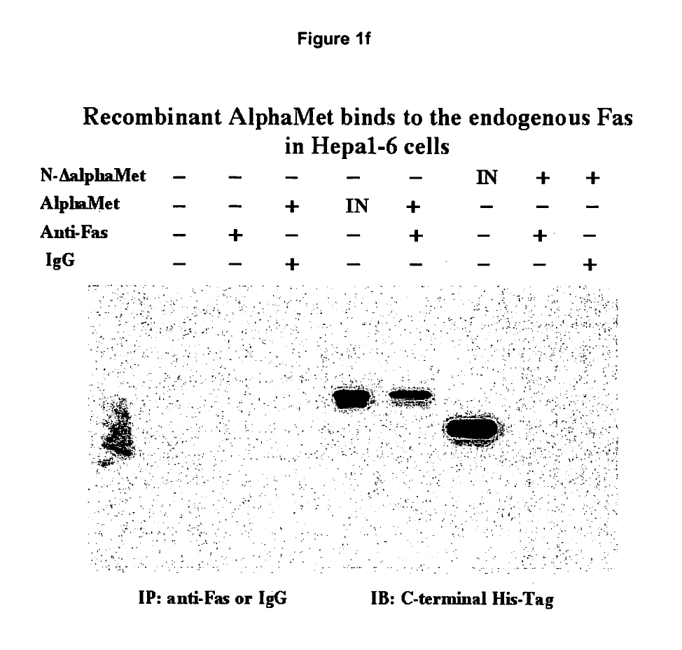

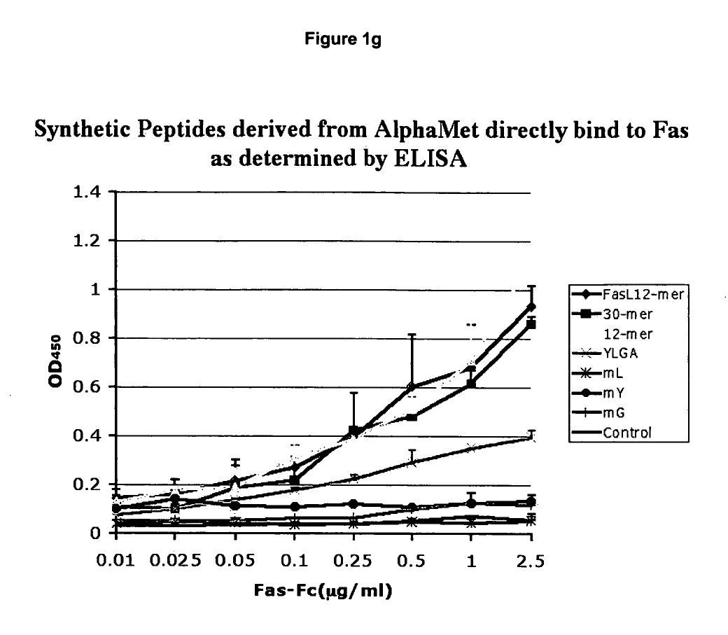

In a previous report using Fas-Fc chimeric construct and ³⁵S-radiolabeled in vitro synthesized mouse Met molecules and pull down assays with protein-A agarose, the extracellular portion of Met was shown to be sufficient for directly binding to the extracellular region of Fas (Wang et al., 2002). To further map the interacting region, expression vectors encoding various portions of the Met molecule were constructed. The alpha and the beta subunit expression vectors encoding the alpha chain (amino acids residues 1 to 306) or the extracellular region of Met beta chain (amino acids 308 to 968 which also contains the transmembrane portion) were analyzed to determine whether both were involved in the interaction with Fas. As shown in FIG. 1a, only Met alpha chain but not the Met beta chain region bound specifically to the Fas molecule (FIG. 1a, left and the right panels, respectively).

The alpha subunit of Met was further analyzed to determine which part of the AlphaMet was crucial for Fas binding. Several N-terminally or C-terminally truncated AlphaMet expression vectors tagged with His-tag were generated (see FIG. 1b) and tested in similar fashion. As depicted in FIG. 1c lane 5, the full length AlphaMet bound Fas very efficiently. C-terminally truncated AlphaMet constructs (amino acid residues 1 to 106 and 1 to 210) also showed robust Fas binding (FIG. 1c, lanes 3 and 4). In contrast, N-terminally truncated AlphaMet lacking the first 100 amino acid residues exhibited no detectable Fas binding (FIG. 1c lane 7). Addition of the short stretch of the transmembrane domain of the Met to either full length or the N-terminally truncated AlphaMet had no effect on their Fas binding ability (FIG. 1c, lanes 6 and 8). These results show that the Fas binding domain in the extracellular region of Met resides within the N-terminal region of the Met alpha chain.

To further explore the underlying reason for AlphaMet and Fas interaction it was hypothesized that some sequence or structural similarity may exist between AlphaMet and Fas/FasL. Various bioinformatics tools were utilized, such as sequence alignment and secondary structure analyses of AlphaMet with Fas and FasL. Interestingly, a 72% identity was found in a seven amino acid stretch in the N-terminal portion of AlphaMet (residues 63 to 70), with that of the FasL (residues 239 to 246 in the C-terminal portion of FasL) (see FIG. 1d). Further analyses indicated that a YLGA motif is well conserved in recently described Met related family members such as Plexin 4A (see discussion below). It was also conserved in the TNF family members, including TNF alpha and TRAIL (FIG. 1d). For a review on the structure-function attributes of the TNF/TNFR superfamily see Bodmer et al., (2002). It was hypothesized that this motif may be responsible for AlphaMet binding to Fas. Therefore, the YLGA motif was mutated to positively or negatively charged amino acids DHER in the context of the full length AlphaMet (see FIG. 1b), expressed and purified in E. coli, and pull down assays were performed using the Fas-Fc chimeric construct (FIG. 1e). YLGA mutated AlphaMet totally lost its ability to bind to Fas, whereas the wild type AlphaMet bound avidly (FIG. 1e). As expected, the N-terminally truncated AlphaMet having residues lacking the first 100 amino acid residues did not bind to Fas.

To further confirm that the N-terminal portion of AlphaMet interacts with Fas, the wild type, YLGA-mutated full length AlphaMet (residues 1 to 306) and the N-terminally deleted AlphaMet (having residues 100 to 306) were directly added to Jurkat cells and Hepa 1-6 cells which express high levels of endogenous Fas. Immunoprecipitation was then performed using anti-Fas antibody or control IgG followed by Western blot analysis using anti-His-tagged antibody as a probe. As predicted, only wild type but not the mutant AlphaMet could be co-precipitated with anti-Fas (FIG. 1f and g).

Given the fact that mutation of YLGA in AlphaMet completely nullified its Fas binding capacity and that the predicted structure of AlphaMet and FasL indicated the YLGA (SEQ ID NO:1) motif would be on the surface in a linear fashion and accessible for interaction, it was possible that short polypeptides harboring the YLGA (SEQ ID NO:1) sequence would be sufficient for Fas binding. Several polypeptides were synthesized that corresponded to the YLGA (SEQ ID NO:1) region of the AlphaMet and they were tested for Fas binding. In the ELISA assays, a fixed amount of a given polypeptide was coated into the wells of a 96-well plate and Fas-Fc was captured and quantified. Also, a fixed amount of Fas-Fc was coated into the wells of a 96-well plate and increasing amounts of coated polypeptides were quantified. Initially peptides of 36 mer (FTAETPIQNVVLHGHHIYLGATNYIYVLNDKDLQKV, SEQ ID NO:25), 12 mer (HHIYLGATNYIY, SEQ ID NO:26), and 4 mer (YLGA, SEQ ID NO:1) in length were tested. These polypeptides bound very specifically and efficiently to Fas although the 4 mer YLGA (SEQ ID NO:1) had reduced activity (FIG. 1h). Additional 4 mer polypeptides were synthesized in which Y, L, or G was replaced by R (i.e. RLGA (SEQ ID NO:27), YRGA (SEQ ID NO:28), and YLRA (SEQ ID NO:29)), a conservative substitution like Y to F (FLGA SEQ ID NO:32) or completely scrambled into four different amino acids to identify whether a single residue was involved. Also, a 13 mer corresponding to the YLGA region of FasL was synthesized (AHSSYLGAVFNLT SEQ ID NO:30). The 36 mer, 12 mer, and the YLGA-13 mer polypeptides corresponding to the sequence of FasL bound Fas strongly, although the binding capacity of the YLGA 4 mer was lower compared to the YLGA 12 mer. It was, however, still significantly higher than that of the scrambled polypeptides (P=0.0019) or the mutated YLGA tetramers (P=0.02), indicating that the neighboring amino acids are important to keep the YLGA motif in a correct conformation for Fas binding. The R replaced YLGA polypeptides (RLGA (SEQ ID NO:27), YRGA (SEQ ID NO:28), and YLRA (SEQ ID NO:29)) completely lost their Fas binding capacity. However, FLGA tetramer exhibited identical binding activity to that of the YLGA tetramer indicating that Y can be substituted with F without loss of activity. Another 13-mer derived from PlexinA4 (TGHIYLGAVNRIY SEQ ID NO:31) also exhibited strong Fas-binding activity, as did a 12-mer derived from the human FasL protein (HHIFLGATNYIY, SEQ ID NO:33). These data indicate that YLGA (SEQ ID NO:1) is not only essential but also sufficient to bind Fas, although the adjacent amino acids enhanced its binding capacity, and the YLGA (SEQ ID NO:1) or FLGA (SEQ ID NO:32) motif worked as an intact entity in Fas binding. These findings identified the region involved in Met and Fas interaction, and led to the discovery of a novel motif in FasL that is sufficient for Fas binding, which was not recognized previously.

### Example 2

This example demonstrates that AlphaMet competes with FasL for binding to Fas.

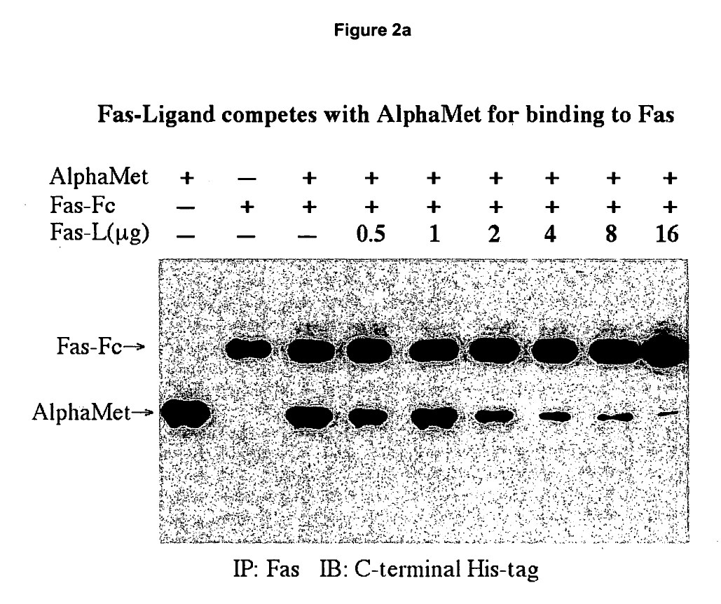

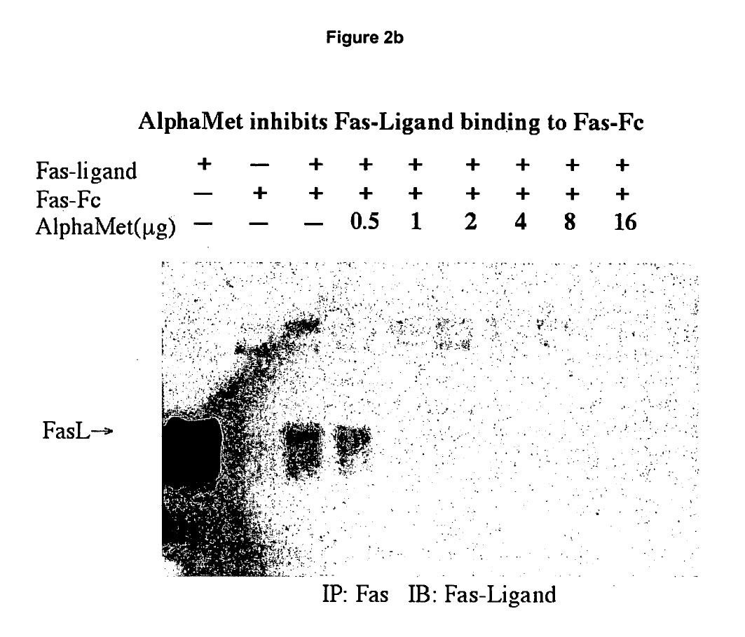

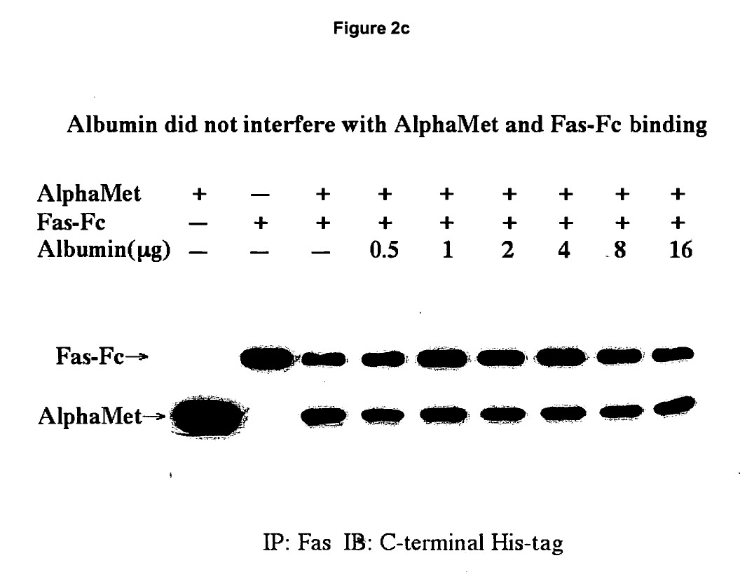

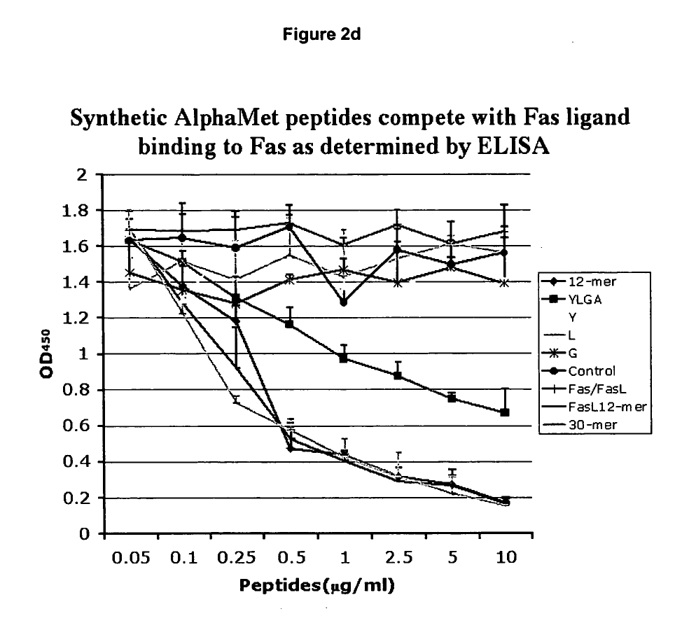

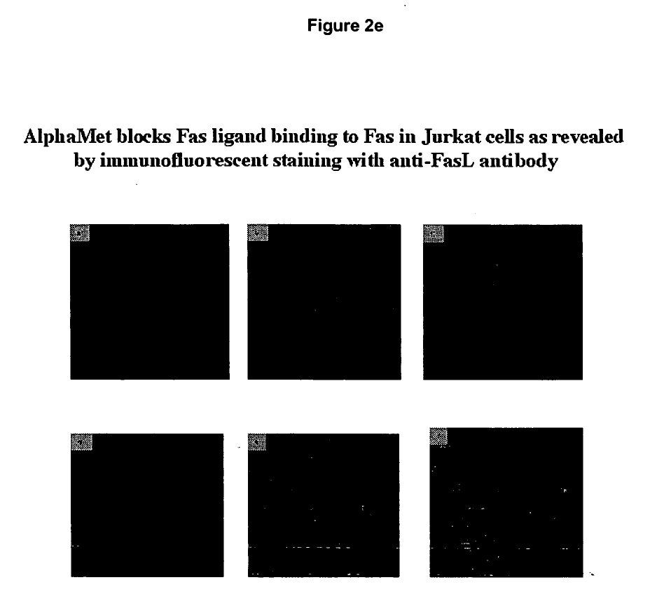

Given the data described above it was possible that AlphaMet could compete with FasL for binding to Fas. Binding and pull down assays were performed using Fas-Fc, recombinant full length AlphaMet, and recombinant FasL. As shown in FIG. 2a and 2c, the binding of AlphaMet to Fas was completely abrogated by increasing amounts of FasL but not unrelated proteins, respectively. In complementary experiments, the addition of increasing amounts of AlphaMet blocked the FasL binding to Fas (FIG. 2b). These results indicate that AlphaMet competes with FasL for Fas binding.

Abrogation of Fas-FasL binding by the presence of the synthetic polypeptides corresponding to the AlphaMet YLGA (SEQ ID NO:1) motif was then analyzed. As shown in FIG. 2d, the binding of FasL to Fas was completely abrogated by increasing amounts of the wild type but not mutated polypeptides corresponding to the YLGA (SEQ ID NO:1) motif of AlphaMet. No significant difference between the 36 mer, 12 mer, and the 13 mer derived from FasL was noted (FIG. 2d), and they all significantly inhibited FasL binding (P=0.0001) as did the 13-mer derived from Polexin A4 and the 12-mer derived from the human FasL protein (SEQ ID NOs: 31 and 33). However, the YLGA tetra-polypeptide presented lower, yet significant inhibition as compared to the control or its mutated counterparts (P=0.0007). The mutated YLGA tetramer and the control polypeptide did not affect the Fas-Fc and Fas-Ligand binding (FIG. 2d). In other experiments, these polypeptides did not block the association between Fas-Fc and its monoclonal mouse anti-Fas antibody, Jo2 (data not shown), suggesting that Fas-Ligand and Jo2 may recognize and bind to different site(s) of Fas. To determine whether AlphaMet or the corresponding synthetic polypeptides bind to Fas and inhibit FasL binding to Fas on intact cells, immunocytostaining experiments were performed using Jurkat cells which express high levels of Fas. As depicted in FIG. 2e, cell surface FasL binding to Fas was blocked by the presence of AlphaMet and synthesized YLGA polypeptides. In these experiments, Jurkat cells were treated with 1 μg/ml of human recombinant FasL at 4° C. for 30 minutes in the presence or absence of recombinant wild type or YLGA mutated AlphaMet or synthetic polypeptides as indicated. The cells were fixed and stained for FasL using anti-FasL antibody.

### Example 3

This example demonstrates that the inventive polypeptides suppress Fas molecule assembly on the cell membrane.

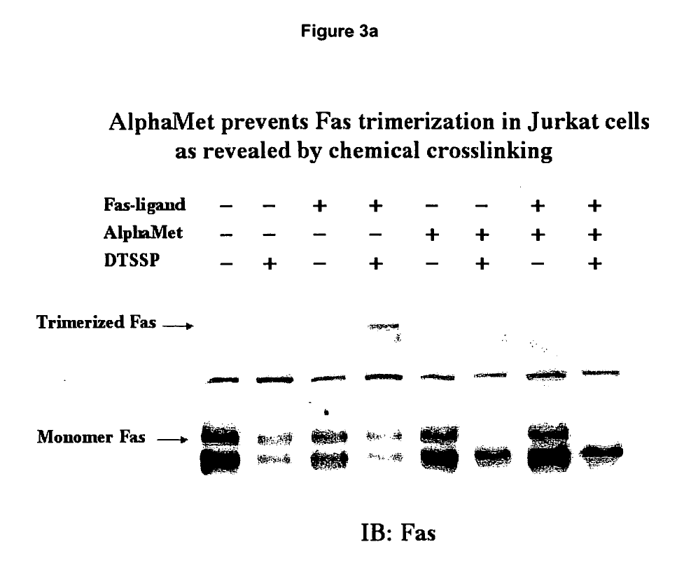

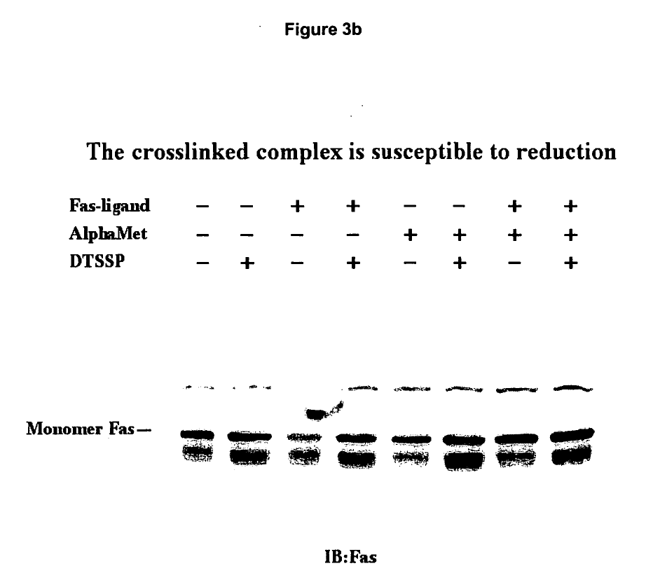

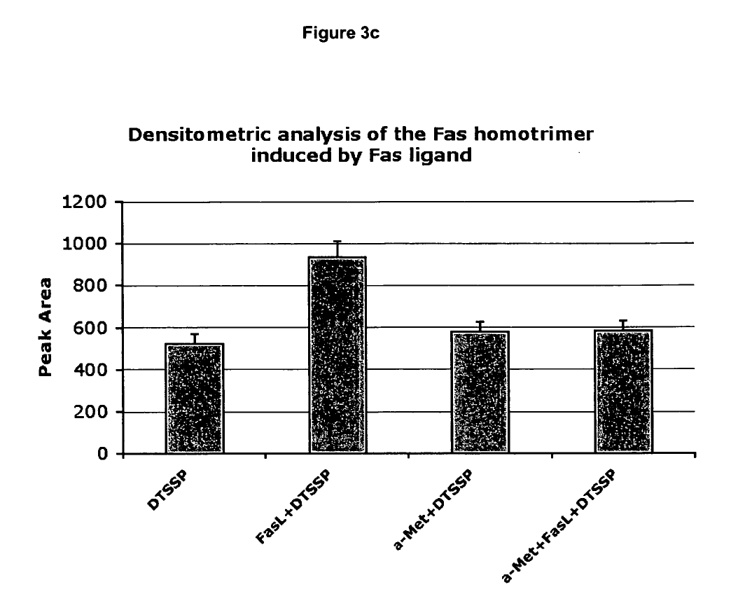

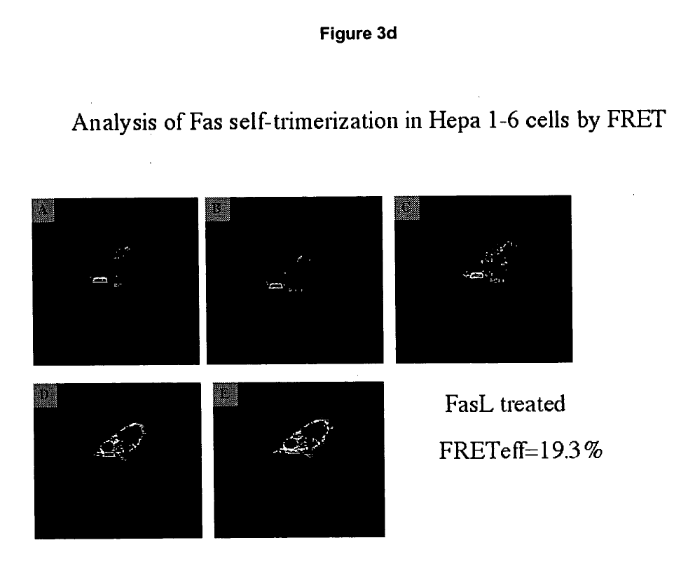

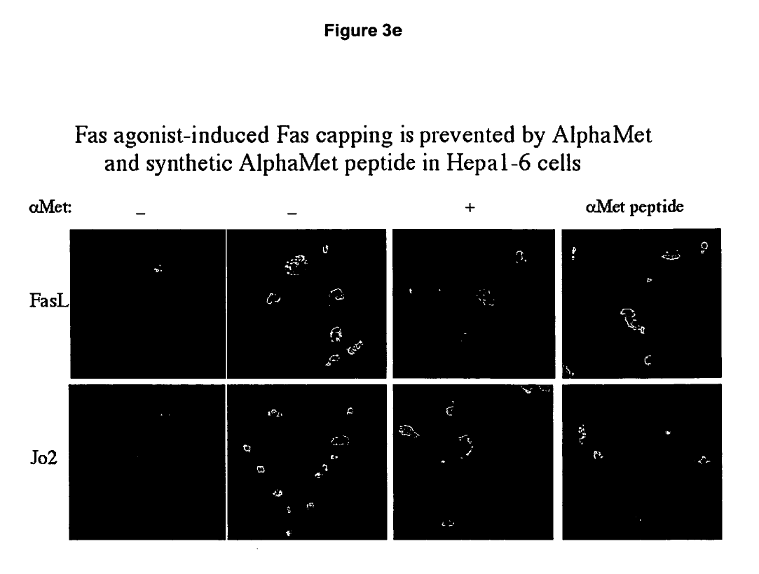

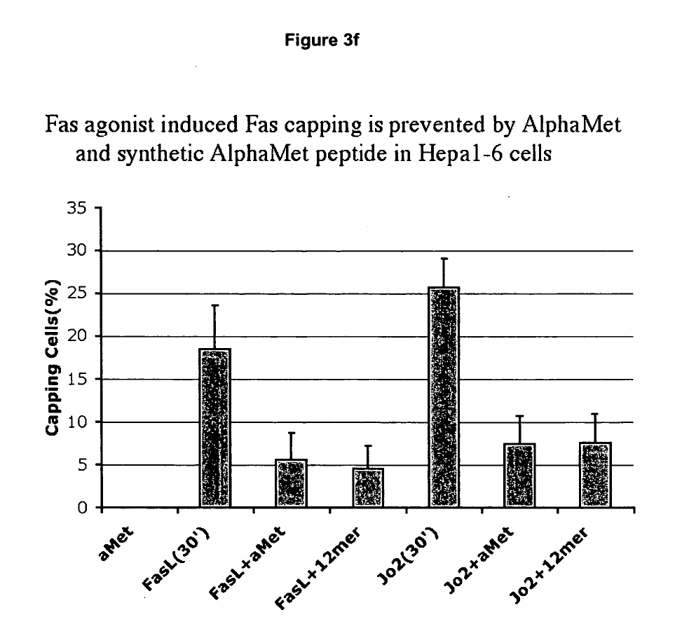

Pre-assembly of Fas is a suspected prerequisite for efficiently initiating Fas signaling. Trimeric Fas exists in some hematopoietic cell lines prior to and independent of Fas ligand binding. Fas mainly exists in monomeric form in hepatic and Jurkat cells (FIG. 3a). It was possible that Fas trimerization process would be affected by AlphaMet. Chemical crosslinking experiments were performed on Jurkat cells and a Western blot using anti-Fas antibody to detect Fas trimerization status was utilized to detect Fas. Thiol-cleavable, membrane-impermeant chemical cross-linker 3,3′dithiobis[sulfosuccinimidylpropionate] (DTSSP) was then applied. Indeed, it was observed that the monomer Fas is the major form of Fas in the Jurkat cell line (FIG. 3a). In the presence of Fas-Ligand and DTSSP a specific high molecular mass (Mr of about 160) complex was found in Jurkat cells which increased about 3 fold in intensity as compared to the ligand unstimulated crosslinked cells (FIG. 3a and 3c). The addition of AlphaMet reduced the Fas trimerization to the background level (FIG. 3a and 3c). When the crosslinked samples were subjected to SDS-PAGE under reducing conditions to break the bonds produced by the crosslinking agent, the trimeric Fas complex disappeared (see FIG. 3a, lane 8). The fact that no crosslinked complex consisting of the Alpha-Met (Mr of about 30,000) and Fas monomers (Mr 50,000) was detected may be because such a complex is undetectable by the monoclonal anti-Fas antibody due to masking the antigenic sites by the bound AlphaMet. Indeed, the amount of the Fas monomer was drastically lower in AlphaMet treated crosslinked samples (see FIG. 3a, lanes 6 and 8).

Intact cells were then used to demonstrate that AlphaMet affects Fas assembly (self-trimerization and clustering). Fluorescence protein tagged Fas molecules and Hepa 1-6 cells were used for these experiments. These cells were transiently co-transfected with CFP and YFP chimeric Fas expression vectors using the lipofectamine 2000 method (Invitrogen). After 24 hours of transfection, about 50% of the cells were fluorescing. The cells were treated by Fas-ligand in the presence or absence of AlphaMet for 30 minutes. Treated cells were then fixed with cold methanol and subjected to FRET analysis. Cells were considered positive if FRET efficiency was over 10%. FRET phenomenon was observed only in cells that were treated with Fas-ligand. The addition of AlphaMet inhibited the FasL-induced FRET phenomenon (FIG. 3d). In these experiments the percent of Fas capping was also determined. The percent of cells exhibiting capping of the fluorescence protein tagged (CFP/YFP) Fas molecules in the presence or absence of FasL and with or without AlphaMet and YLGA polypeptides was enumerated. As shown in FIG. 3e and FIG. 3f, Fas capping occurred efficiently and markedly within 30 minutes treatment of FasL or Fas antagonist Jo2 monoclonal antibody (20% and 30% of fluorescent cells showed Fas capping, respectively) and this phenomenon was significantly (P=0.006 for FasL) inhibited to the background level by AlphaMet and AlphaMet derived 12 mer YLGA polypeptide. Interestingly, Jo2 mediated Fas clustering was also inhibited (P=0.0016 for Jo2 treated samples) indicating that trimerization of the receptor is prohibited when bound to AlphaMet or YLGA polypeptide.

### Example 4

This example demonstrates that the inventive polypeptides protect against apoptosis in vitro.

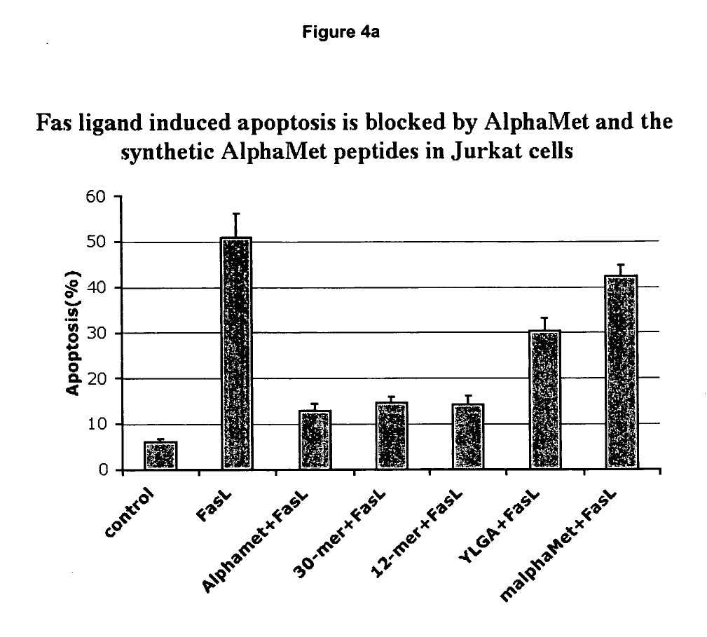

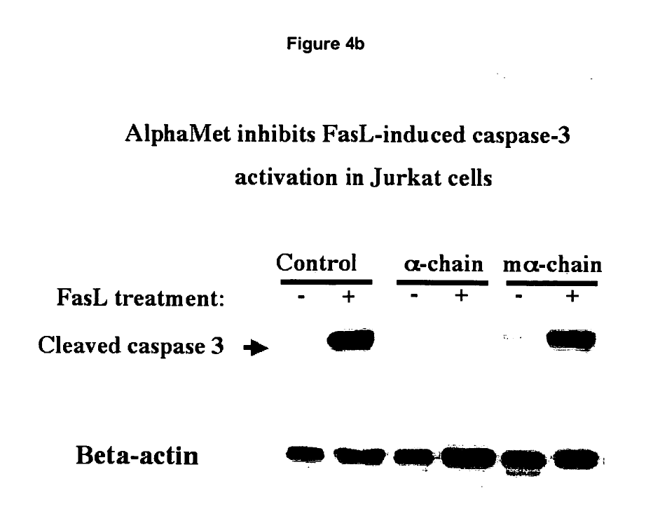

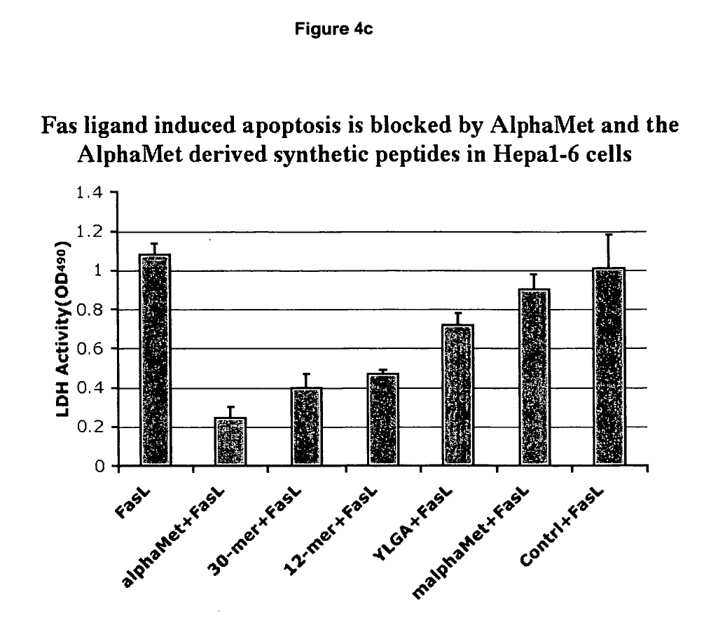

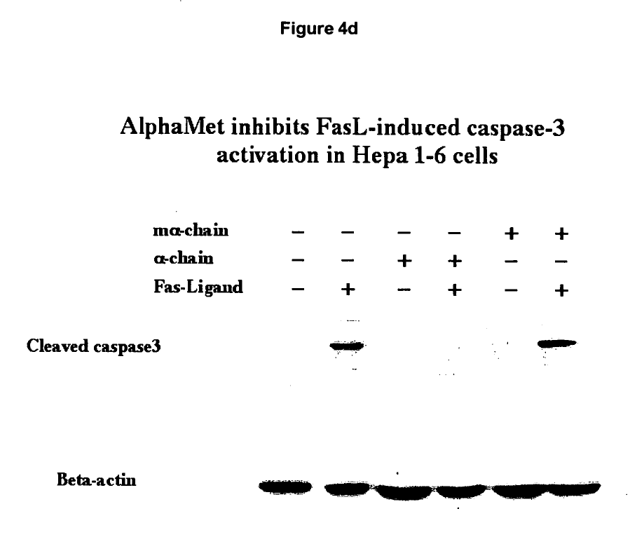

As mentioned above, Jurkat cells are one of the most widely used cell lines in Fas-mediated apoptosis investigation due to their high sensitivity. It was determined that these cells express very low levels of Met as evident by Western blot when compared to hepatic cells such as Hepa 1-6. The major form of Met observed in Jurkat cells was a p110 size form as opposed to the well known p145 form determined by two different antibodies against Met beta chain (c28, a polyclonal antibody that recognizes the C-terminal end of the beta chain, and DL-21, a monoclonal antibody that recognizes the extracellular portion of the beta chain). The high sensitivity of these cells to Fas killing as opposed to hepatic cells which express high levels of Met and are relatively resistance to Fas killing without sensitization (i.e. inhibition of RNA or protein synthesis) was likely due to low Met expression. Thus, it was examined whether recombinant AlphaMet protects Jurkat cells from Fas-ligand induced apoptosis. Apoptosis in Jurkat cells was induced by recombinant human Fas-ligand. As shown in FIG. 4a, purified recombinant AlphaMet significantly reduced the death rate from about 50% to 12% which is close to the background level. Flow cytometry analyses of the Propidium Iodide stained Jurkat cells indicated the sub-G1 population was reduced to 8% in the Fas-ligand with AlphaMet group, compared to the 48% apoptotic proportion in the Fas-ligand treated control. The YLGA motif mutated recombinant AlphaMet protein did not have any effects on the rate of apoptosis. The synthetic YLGA polypeptide blocked the Fas ligand induced apoptosis efficiently (see FIG. 4a) which is consistent with other binding experiments.

To provide biochemical evidence that AlphaMet inhibited FasL-mediated apoptosis, the activation of Caspase 3, a down stream executioner in Fas mediated apoptosis, was analyzed in apoptotic Jurkat cells. As shown in FIG. 4b, Caspase 3 activation (cleavage to a 17 kDa active form which is recognized by a specific monoclonal antibody) occurred after Fas-ligand addition. The extent of Caspase 3 activation was markedly reduced by wild type but not YLGA motif mutated recombinant AlphaMet protein (FIG. 4b).

A similar set of experiments described above were also carried out using Hepa 1-6 cells which are adherent cells. As shown in FIG. 4c, recombinant AlphaMet protein and YLGA containing peptides (12 mer, HHIYLGATNYIY, SEQ ID NO:26; and 4 mer, YLGA, SEQ ID NO:1) significantly inhibited FasL induced apoptosis in these cells while mutated YLGA motif had no effect. Activation of caspase-3 was also inhibited by the wild type but not the YLGA mutated AlphaMet (FIG. 4d). Interestingly, apoptosis induced by the Fas agonist Jo2 was also efficiently inhibited by the addition of exogenous recombinant wild type AlphaMet. Stable cell lines form Hepa 1-6 engineered to express AlphaMet also exhibited dramatic resistance to Fas killing as compared to control vector transfected clones. The results were consistent with those from the Jurkat cells (FIG. 4d). Since the YLGA (SEQ ID NO: 1) motif did not block the binding of Jo2 to Fas, yet protected the Hepa 1-6 cells from Jo2 induced apoptosis, it is conceivable that the binding of Fas to AlphaMet or YLGA polypeptide blocks Fas trimerization and clustering, thus Fas activation. In support of this notion, reduced Jo2-induced Fas capping by AlphaMet and 12 mer YLGA was observed (see FIG. 3f).

### Example 5

This example demonstrates that inventive polypeptides protect against apoptosis in vivo.

It is well known in the literature that the liver is one of the most sensitive organs to Fas mediated apoptosis. Injection of the Fas agonist Jo2 antibody, which causes mice to experience Fas aggregation resulting in massive hepatocyte apoptosis, fulminant hepatic failure, and death within hours. This model was used to test if AlphaMet and the YLGA polypeptides could protect livers from Fas-induced apoptosis. Given the fact that AlphaMet blocked Fas trimerization and Fas micro-aggregation, and the fact that Fas oligomerization is essential in Fas signaling, it was reasonable to suspect that AlphaMet could block the Fas mediated apoptosis in vivo. Balb/c mice were used in these experiments. Groups of animals (n=10) were injected systemically with Jo2 (0.25 μg/g of body weight) with or without purified recombinant AlphaMet, synthetic 12 mer YLGA polypeptide (HHIYLGATNYIY, SEQ ID NO:26), or their control counterparts (N-terminally truncated AlphaMet, YLGA mutated recombinant AlphaMet or scrambled 12 mer polypeptide) as described in the materials and methods. Mice were sacrificed within 6 hours or sooner if they were moribund and their livers were subjected to biochemical determination of Caspase-3 activity and histological analyses (i.e. TUNEL staining).

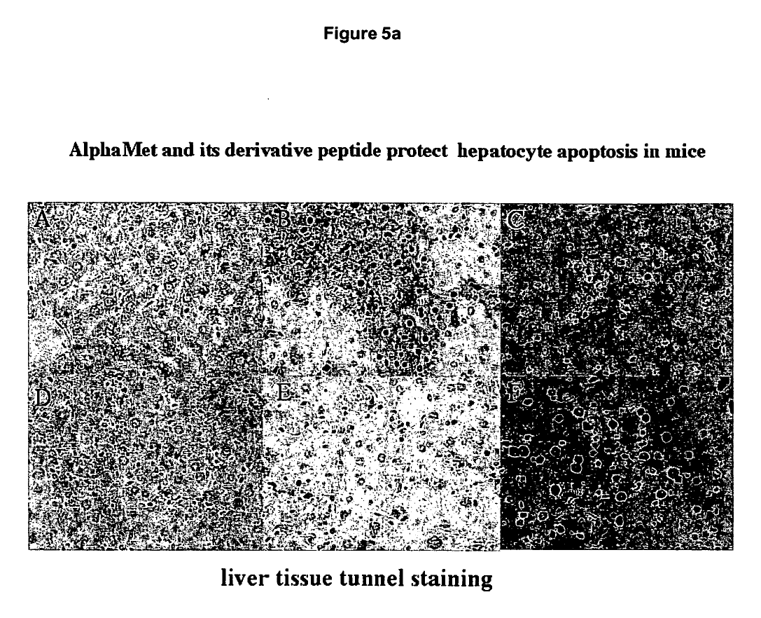

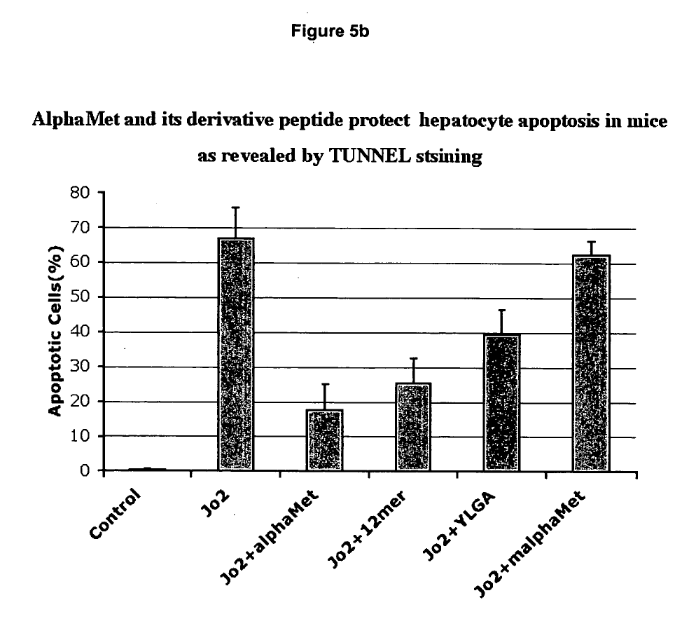

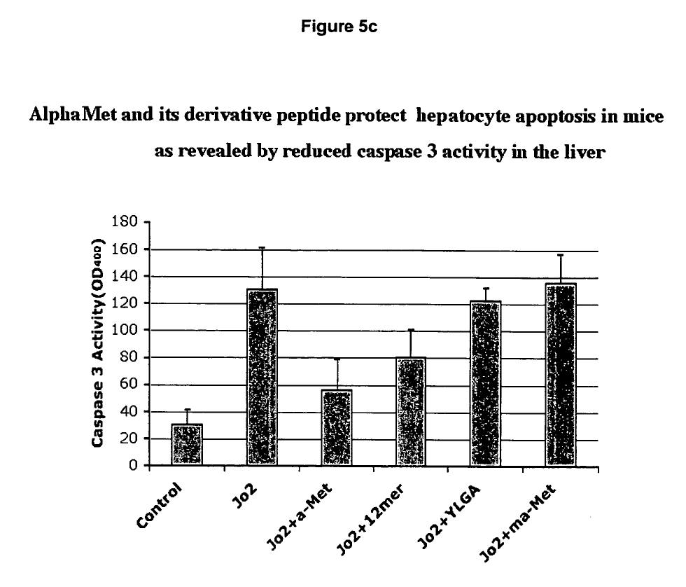

These studies were done in a blind manner. The mice that were injected with Jo2 only or Jo2 plus mutated AlphaMet showed massive hemorrhagic livers, which was evident grossly as well as microscopically, whereas the AlphaMet treated or the YLGA polypeptide treated animals showed little or no sign of hemorrhage and their livers were normal in appearance. Determination of the extent of apoptosis by TUNEL staining revealed that the percentage of apoptotic cells in the Jo2 treated group was 67% (untreated controls were less than one percent), which was drastically reduced to 18% (ANOVA, P=0.009) by AlphaMet addition, to 25% by the 12 mer (P=0.019), and to 40% by the YLGA tetramer (P=0.087). Mutated AlphaMet did not change the rate of apoptosis (FIG. 5a and b).

Analyses of the liver lysates for enzymatic activity of caspase-3 (FIG. 5c) indicated that a significant reduction occurred in the activity of Caspase 3 in AlphaMet (P<0.001, ANOVA) and YLGA 12 mer polypeptide (P<0.001 ANOVA) treated groups compared to that of the Jo2 only group. The YLGA mutated control, however, did not affect the caspase-3 activity in comparison to the Jo2 group. These results indicated that the AlphaMet, as well as the 12 mer YLGA polypeptide, are potent agents in suppressing the Fas-mediated apoptosis in vivo.

### Bibliography

i. Algeciras-Schimnich et al., “Molecular Ordering of the Initial Signaling Events of CD95”, Mol. Cell. Biol., 22, 207-222 (2002).
ii. Arakaki et al., “Hepatocyte growth factor/scatter factor activates the apoptosis signaling pathway by increasing caspase-3 activity in sarcoma 180 cells”, Biochem Biophys Res Commun., 245(1), 211-215 (1998).
iii. Arakaki et al., “Involvement of oxidative stress in tumor cytotoxic activity of hepatocyte growth factor/scatter factor”, J Biol Chem, 274(19), 13541-13546. (1999).
iv. Banner et al., “Crystal structure of the soluble human 55 kd TNF receptor-human TNF beta complex: implications for TNF receptor activation”, Cell, 73(3), 431-445 (1993).
v. Blume-Jensen et al., “Oncogenic kinase signaling”, Nature, 411(6835), 355-365 (2001).
vi. Bodmer et al., “The molecular architecture of the TNF superfamily”, Trends in Biochemical Sciences, 27(1), 19-26 (2002).
vii. Cha et al., “2.8-Å resolution crystal structure of human TRAIL, a cytokine with selective antitumor activity”, Immunity, 11, 253-261 (1999).
viii. Chan et al., “A domain in TNF receptors that mediates ligand-independent receptor assembly and signaling”, Science, 288(5475), 2351-2354 (2000).
ix. Comoglio et al., “Plasminogen-related growth factor and semaphorin receptors: a gene superfamily controlling invasive growth”, Exp Cell Res., 253(1), 88-99 (1999).
x. Conner et al., “HGF-mediated apoptosis via p53/bax-independent pathway activating JNK1”, Carcinogenesis, 20(4), 583-590 (1999).
xi. Connolly et al., “In vivo inhibition of fas ligand-mediated killing by TR6, a fas ligand decoy receptor”, Pharm. and Exp. Therapeutics, 298, 25-33 (2001).
xii. Cremesti et al., “Ceramide Enables Fas to Cap and Kill”, J. Biol. Chem., 276, 23954-23961 (2001).
xiii. Gherardi et al., “A Bandyopadhyay, G Hartmann, and P. J. G. Butler. Functional map and domain structure of MET, the product of the c-met protooncogene and receptor for hepatocyte growth factor/scatter factor”, PNAS, 100, 12039-12044 (2004).
xiv. Giordano et al., “Tyrosine kinase receptor indistinguishable from the c-met protein”, Nature, 339(6220), 155-156 (1989)
xv. Gohda et al., “Induction of apoptosis by hepatocyte growth factor/scatter factor and its augmentation by phorbol esters in Meth A cells”, Biochem Biophys Res Commun., 245(1), 278-283 (1998).
xvi. Holler et al., “Two adjacent trimeric fas ligands are required for fas signaling and formation of a death-inducing signaling complex”, Mol. Cell. Biol., 23, 1428-1440 (2003).
xvii. Hymowitz et al., “Triggering cell death: the crystal structure of Apo2L/TRAIL in a complex with death receptor 5”, Mol Cell, 4(4), 563-71 (1999).
xviii. Jones et al., “Structure of tumor necrosis factor”, Nature, 338, 225-228 (1989).
xix. Komada et al., “Proteolytic processing of the hepatocyte growth factor/scatter factor receptor by furin”, FEBS Lett., 328(1-2), 25-29 (1993).
xx. Liu et al., “Ligand-receptor binding revealed by the TNF family member TALL-1”, Nature, 423(6935), 49-56 (2003).
xxi. Locksley et al., “The TNF and TNF Receptor Superfamilies: Integrating Mammalian Biology”, Cell, 104, 487-501 (2001).
xxii. Muppidi et al., “Ligand-independent redistribution of Fas (CD95) into lipid rafts mediates clonotypic T cell death”, Nature Immunology, 5, 182-189 (2004).
xxiii. Nisihara et al., “Humanization and epitope mapping of neutralizing anti-human Fas Ligand monoclonal antibodies: structural insights into Fas/Fas Ligand interaction”, J. Immun., 167, 3266-3275 (2001).
xxiv. Orlinick et al., “Requirement of Cysteine-rich Repeats of the Fas Receptor for Binding by the Fas Ligand”, J Biol Chem., 272(46), 28889-28894 (1997).
xxv. Roschke et al., “BLyS and APRIL form biologically active heterotrimers that are expressed in patients with systemic immune-based rheumatic diseases”, J. Immunol., 169, 4314-4321 (2002).
xxvi. Siegel et al., “Fas preassociation required for apoptosis signaling and dominant inhibition by pathogenic mutations”, Science, 288(5475), 2354-7 (2000).
xxvii. Stamos et al., “Crystal structure of the HGF beta-chain in complex with the Sema domain of the Met receptor”, EMBO Journal, 23, 2325-35 (2004).
xxviii. Starling et al., “Identification of Amino Acid Residues Important for Ligand Binding to Fas”, J. Exp. Med., 185(8), 1487-1492 (1997).
xxix. Thatte et al., “Apoptosis: clinical relevance and pharmacological manipulation”, Drugs, 54(4), 511-32 (1997).
xxx. Tibbetts et al., “The death effector domain protein family: regulators of cellular homeostasis”, Nature Immunology, 4, 404-409 (2003).
xxxi. Trusolino et al., “Scatter-factor and semaphorin receptors: cell signaling for invasive growth”, Nature Reviews Cancer, 2, 289-300 (2002).
xxxii. Wallach et al., “Tumor necrosis factor receptor and fas signaling mechanisms”, Annual Review of Immunology, 17, 331-367 (1999).
xxxiii. Wang et al., “A mechanism of cell survival: sequestration of Fas by the HGF receptor Met”, Mol Cell, 9(2), 411-21 (2002).
xxxiv. Wolf et al., “Interaction with TrkA Immobilizes gp75 in the High Affinity Nerve Growth Factor Receptor Complex”, J. Biol. Chem., 270, 2144-2138 (1995).

These references and all other references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A synthetic polypeptide that attenuates the activation of Fas or TNFR1 or both Fas and TNFR1, which consists of between about 4 and about 50 amino acids.

2. The synthetic polypeptide of claim 1, which comprises 4 contiguous amino acid residues having the sequence YLGA (SEQ ID NO:1), YLGG (SEQ ID NO:2), or FLGA (SEQ ID NO: 32).

3. The synthetic polypeptide of claim 1, which comprises 5 contiguous amino acid residues having the sequence IYLGA (SEQ ID NO:3), IYLGG (SEQ ID NO:4), YLGAV (SEQ ID NO:5), YLGGV (SEQ ID NO:6), IFLGA (SEQ ID NO:35), IFLGG (SEQ ID NO:36), FLGAV (SEQ ID NO:37), or FLGGV (SEQ ID NO:38).

4. The synthetic polypeptide of claim 1, which comprises 6 contiguous amino acid residues having the sequence IYLGAV (SEQ ID NO:7), IYLGGV (SEQ ID NO:8), IFLGAV (SEQ ID NO:39), or IFLGGV (SEQ ID NO:40).

5. A synthetic polypeptide comprising between about 4 and about 50 amino acids, which comprises 4 contiguous amino acid residues having the sequence YLGA (SEQ ID NO:1), YLGG (SEQ ID NO:2), or FLGA (SEQ ID NO: 32).

6. The synthetic polypeptide of claim 5, which comprises 5 contiguous amino acid residues having the sequence IYLGA (SEQ ID NO:3), IYLGG (SEQ ID NO:4), YLGAV (SEQ ID NO:5), YLGGV (SEQ ID NO:6), IFLGA (SEQ ID NO:35), IFLGG (SEQ ID NO:36), FLGAV (SEQ ID NO:37), or FLGGV (SEQ ID NO:38).

7. The synthetic polypeptide of claim 5, which comprises 6 contiguous amino acid residues having the sequence IYLGAV (SEQ ID NO:7), IYLGGV (SEQ ID NO:8), IFLGAV (SEQ ID NO:39), or IFLGGV (SEQ ID NO:40).

8. The polypeptide of any of claims 1-7, consisting of less than about 15 amino acids.

9. A polypeptide consisting essentially of a C-terminal truncation of the alpha subunit of the Met receptor that attenuates the activation of Fas, TNFR1 or both Fas and TNFR1.

10. The polypeptide of claim 9, which comprises from about amino acid 1 to about amino acid 306 of the alpha subunit of the Met receptor.

11. The polypeptide of claim 9, which comprises from about amino acid 1 to about amino acid 210 of the alpha subunit of the Met receptor.

12. The polypeptide of claim 9, which comprises from about amino acid 1 to about amino acid 106 of the alpha subunit of the Met receptor.

13. The polypeptide of any of claims 1, 5, or 9, which attenuates the activation of both Fas and TNFR1.

14. The synthetic polypeptide of any of claims 1, 5, or 9, which attenuates the activation of Fas, TNFR1 or both Fas and TNFR1 in vivo.

15. The polypeptide of any of claims 1, 5, or 9, which attenuates the activation of Fas, TNFR1 or both Fas and TNFR1 in vitro.

16. The polypeptide of any of claims 1, 5, or 9 in a substantially pure or recombinant form.

17. A method of attenuating cell death comprising administering to a population of cells a polypeptide of any of claims 1, 5, or 9 in an amount sufficient to attenuate cell death within the population of cells.

18. The method of claim 18, wherein said cell death is apoptosis.

19. The method of claim 18, wherein said attenuation occurs in vitro.

20. The method of claim 18, wherein said attenuation occurs in vivo.

21. A method of attenuating inflammation with a patient comprising administering to a patient at risk for inflammation a polypeptide of any of claims 1, 5, or 9 in an amount and at a location sufficient to attenuate inflammation within the patient.

22. A method of treating liver disease, kidney disease, disorders of the pancreas, autoimmune diseases such as AIDS, and neurodegenerative disorders such as Alzheimer's or Parkinson's within a patient in need of such treatment, comprising administering to the patient a polypeptide of any of claims 1, 5, or 9 in an amount and at a location sufficient to treat the disease within the patient.

23. The method according to claim 22, wherein the disorder is liver failure.

24. The method according to claim 22, wherein the disorder is hepatitis.

25. The method according to claim 22, wherein the disease is an autoimmune disease.

26. The method according to claim 25, wherein said autoimmune disease is rheumatoid arthritis.

27. The method according to claim 22, wherein said disorder is Alzheimer's disease.

28. A pharmaceutical composition comprising a polypeptide of any of claims 1, 5, or 9 and a pharmaceutically-acceptable carrier.

29. The pharmaceutical composition of claim 28, formulated for parenteral administration.

30. The pharmaceutical composition of claim 28, formulated for oral administration.

31. The pharmaceutical composition of claim 28, formulated for topical administration.

32. A pharmaceutical composition comprising the polypeptide of claim 8 and a pharmaceutically-acceptable carrier.

33. The pharmaceutical composition of claim 32, formulated for parenteral administration.

34. The pharmaceutical composition of claim 32, formulated for oral administration.

35. The pharmaceutical composition of claim 32, formulated for topical administration.


SEQUENCE LISTING (40 sequences)

SEQ ID NO: 1 - protein, 4 aa, Artificial
  Synthetic Peptide
YLGA

SEQ ID NO: 2 - protein, 4 aa, Artificial
  Synthetic Peptide
YLGG

SEQ ID NO: 3 - protein, 5 aa, Artificial
  Synthetic Peptide
IYLGA

SEQ ID NO: 4 - protein, 5 aa, Artificial
  Synthetic Peptide
IYLGG

SEQ ID NO: 5 - protein, 5 aa, Artificial
  Synthetic Peptide
YLGAV

SEQ ID NO: 6 - protein, 5 aa, Artificial
  Synthetic Peptide
YLGGV

SEQ ID NO: 7 - protein, 6 aa, Artificial
  Synthetic Peptide
IYLGAV

SEQ ID NO: 8 - protein, 6 aa, Artificial
  Synthetic Peptide
IYLGGV

SEQ ID NO: 9 - DNA, 18 bp, Artificial
  Primer
atgaaggctcccaccgtg

SEQ ID NO: 10 - DNA, 21 bp, Artificial
  Primer
atggggactgcagcagcaaag

SEQ ID NO: 11 - DNA, 20 bp, Artificial
  Primer
atgccacaagggaagaagtg

SEQ ID NO: 12 - DNA, 19 bp, Artificial
  Primer
ctttgctgctgcagtcccg

SEQ ID NO: 13 - DNA, 18 bp, Artificial
  Primer
cagtgaataaccaggagg

SEQ ID NO: 14 - DNA, 24 bp, Artificial
  Primer
tctcttccttcttttttctgtcag

SEQ ID NO: 15 - DNA, 19 bp, Artificial
  Primer
attctgatccggttgaacg

SEQ ID NO: 16 - DNA, 18 bp, Artificial
  Primer
atgaaggctcccaccgtg

SEQ ID NO: 17 - DNA, 35 bp, Artificial
  Primer
cgggatcgatcacgaacgcacaaactacatttatg

SEQ ID NO: 18 - DNA, 34 bp, Artificial
  Primer
cggggatccatggccgtgtaggacgacattctgg

SEQ ID NO: 19 - DNA, 19 bp, Artificial
  Primer
ctttgctgctgcagtcccg

SEQ ID NO: 20 - DNA, 18 bp, Artificial
  Primer
atggaggctcccaccgtg

SEQ ID NO: 21 - DNA, 21 bp, Artificial
  Primer
atggggactgcagcagcaaag

SEQ ID NO: 22 - DNA, 21 bp, Artificial
  Primer
atggccacaagggaagaagtg

SEQ ID NO: 23 - DNA, 26 bp, Artificial
  Primer
tcaatggtgatggtgatgatgaccgg

SEQ ID NO: 24 - protein, 4 aa, Artificial
  Synthetic Peptide
DHER

SEQ ID NO: 25 - protein, 36 aa, Artificial
  Synthetic Peptide
FTAETPIQNVVLHGHHIYLGATNYIYVLNDKDLQKV

SEQ ID NO: 26 - protein, 12 aa, Artificial
  Synthetic Peptide
HHIYLGATNYIY

SEQ ID NO: 27 - protein, 4 aa, Artificial
  Synthetic Peptide
RLGA

SEQ ID NO: 28 - protein, 4 aa, Artificial
  Synthetic Peptide
YRGA

SEQ ID NO: 29 - protein, 4 aa, Artificial
  Synthetic Peptide
YLRA

SEQ ID NO: 30 - protein, 13 aa, Artificial
  Synthetic Peptide
AHSSYLGAVFNLT

SEQ ID NO: 31 - protein, 13 aa, Artificial
  Synthetic Peptide
TGHIYLGAVNRIY

SEQ ID NO: 32 - protein, 4 aa, Artificial
  Synthetic Peptides
FLGA

SEQ ID NO: 33 - protein, 12 aa, Artificial
  Synthetic Peptide
HHIFLGATNYIY

SEQ ID NO: 34 - protein, 4 aa, Artificial
  Synthetic Peptide
FLGG

SEQ ID NO: 35 - protein, 5 aa, Artificial
  Synthetic Peptide
IFLGA

SEQ ID NO: 36 - protein, 5 aa, Artificial
  Synthetic Peptide
IFLGG

SEQ ID NO: 37 - protein, 5 aa, Artificial
  Synthetic Peptide
FLGAV

SEQ ID NO: 38 - protein, 5 aa, Artificial
  Synthetic Peptide
FLGGV

SEQ ID NO: 39 - protein, 6 aa, Artificial
  Synthetic Peptide
IFLGAV

SEQ ID NO: 40 - protein, 6 aa, Artificial
  Synthetic Peptide
IFLGGV